(19) United States
(12) Patent Application Publication
(10) Pub. No.: US 2020/0138971 A1
(43) Pub. Date: May 7, 2020

(54) PROCESS TO DETERMINE EFFICACY OF SINGLE HUMAN ANTIBODY TYPE NANOPARTICLE CONJUGATE

(71) Applicant: THE CURATORS OF THE UNIVERSITY OF MISSOURI, Columbia, MO (US)

(72) Inventors: Raghuraman Kannan, Columbia, MO (US); Ajit Prakash Zambre, Columbia, MO (US); Anandhi Upendran, Columbia, MO (US); Henry White, Columbia, MO (US)

(21) Appl. No.: 15/525,560

(22) PCT Filed: Nov. 13, 2015
(86) PCT No.: PCT/US2015/060645

§ 371 Date: May 9, 2017

Related U.S. Application Data
(60) Provisional application No. 62/123,349, filed on Nov. 13, 2014.

(51) Int. Cl. A61K 47/68; A61K 47/69; A61K 47/60

(52) U.S. Cl. CPC A61K 47/6855; A61K 47/60; A61K 47/6923

(57) ABSTRACT

A preferred embodiment is a method of formation of nanoparticle conjugates for cancer diagnostics, imaging and therapy, wherein the method allows determination a priori the efficacy of the nanoparticle conjugate for treatment of disease. A preferred stable pharmaceutical formulation includes a single antibody agent retaining its functionality in accordance with a predetermination of an a priori efficacy and electrostatically or covalently linked to a nanoparticle in a predetermined ratio in accordance with the a priori efficacy. Preferred embodiments also provide a method for forming single human antibody nanoparticle conjugates. The methods retain properties of the human antibody with a fabrication process that also allows control of the bonding mode of the antibody to the surface of AuNP, such that the bonding mode can be predetermined to be either electrostatic or covalent.

### PRIORITY CLAIM AND REFERENCE TO RELATED APPLICATION

This application claims priority under 35 U.S.C 119 and all applicable statutes and treaties from prior U.S. Provisional Application Ser. No. 62/123,349, which was filed on Nov. 13, 2014.

### FIELD

Fields of the invention include processes to determine efficacy of single human antibody type nanoparticle conjugates, conjugate fabrication and the fabrication of testing, imaging and treatment nanoparticle conjugates. Applications of the invention include the field of diagnosis, imaging and therapy of human cancer. Example specific applications of the invention include treatment of breast cancer.

### BACKGROUND

Despite widespread attention, testing, treatments and therapies, breast cancer still remains a serious and deadly cancer in women. Improving current therapies could potentially extend the lives of thousands of individuals that contract the disease.

Currently accepted diagnoses for breast cancer start with a mammogram screening test, during which an x-ray image of a breast is taken to check for tumors.

Another technique for breast cancer diagnosis is ultrasound imaging. This technique provides a complementary image to that obtained by x-ray imaging.

Other techniques for diagnosing breast cancer include obtaining sample(s) of a suspected volume using needle aspiration for pathology analyses, x-ray computerized axial tomography (CAT) scan, and magnet resonance imaging (MRI).

Post-diagnosis treatments include heightened monitoring and observation, surgical removal of identified tumors, removal of one or more breasts, external-beam radiation therapy, chemotherapy, and brachy therapy. Each of these techniques has some drawbacks, and the shortened life-time and fatality rate for breast cancer remains unacceptably high. There remains a need for new and improved therapeutic modalities, especially for treating inoperable breast cancer.

Typical chemotherapy agents include a stable pharmaceutical formulation comprising pertuzumab, trastuzumab, or other anti-HER2 human antibody agents. These agents deliver a low dose of antibody drug, administered over a period of several months. Difficulties include efficacy for certain tumors, and ability to accurately control dosage.

Human antibody agents have an affinity for human cancer cells.

Human antibody agents have an affinity for cancer vasculature.

Results contained in Katti et al., U.S. Published Patent Application Number 20120134918, show that a suitable pharmaceutical mixture containing Gum Arabic coated ¹⁹⁸Au radioactive gold nanoparticles is an effective theranostic agent for treatment of human prostate cancer. The Gum Arabic serves as a stabilizer. Results also show Gum Arabic coated non-radioactive gold (¹⁹⁷Au) are suitable theranostic human cancer agents.

Gold-198, also denoted herein and also in the literature as ¹⁹⁸Au, or as Au-198, because of its higher energy of emission (βₘₐₓ=0.96 MeV; half-life of 2.7 days) has been used as a permanent implant either alone or as an adjunct to external-beam radiation therapy of cancers. See, e.g., Knight P J, et al., “The use of Interstitial Radiation Therapy in the Treatment of Persistent, Localized, and Unresectable Cancer in Children,” Cancer 57: 951-4 (1986); Rich T. A., “Radiation Therapy for Pancreatic Cancer: Eleven Year Experience at the JCRT,” Int J Radiat Oncol Biol Phys. 11:759-63 (1985). Brachytherapy implants of large radioactive gold seeds provide rapid delivery of radiation at a very high dose rate, thus avoiding some of the radiologic problems associated with iodine. However, because of the high heterogeneity of radioactive ¹⁹⁸Au seeds and liquids, oncologists have developed a consensus that a majority of patients receiving low/high energy brachy therapy will experience post treatment symptoms including adverse side effects to severe clinical complications. Recognized complications for prostate cancer treatment include proctitis, cystitis, incontinence and rectal bleeding. See, e.g., Dall'Era M A, et al, “Hyperbaric Oxygen Therapy for Radiation Induced Proctopathy in Men Treated for Prostate Cancer,” J Urol 176: 87-90 (2006).

The therapeutic nature of Gum Arabic coated ¹⁹⁸Au nanoparticles is due to emission of an energetic electron during radioactive decay of ¹⁹⁸Au (beta decay, where the maximum energy of emission (13.)=0.96 MeV. The half-life of ¹⁹⁸Au=2.7 days). A gamma ray is also emitted by ¹⁹⁸Au during beta decay. The gamma ray can be used for imaging, to obtain information on location and concentration of Gum Arabic ¹⁹⁸Au nanoparticles. ¹⁹⁸Au nanoparticles can be employed to produce a theranostic agent for cancer treatment.

The therapeutic nature of Gum Arabic coated radioactive ¹⁹⁹Au is also due to its emission of an energetic electron during radioactive decay (beta decay, where the maximum energy of emission (βₘₐₓ)=0.452 MeV. The half-life of ¹⁹⁹Au=3.13 days). A gamma ray is also emitted during beta decay. The gamma rays can be used for imaging, to obtain information on location and concentration of Gum Arabic coated ¹⁹⁹Au nanoparticles. The isotope ¹⁹⁹Au emits a 158 KeV gamma ray for scintigraphic imaging properties that is superior to the 412 KeV gamma ray emitted by ¹⁹⁸Au. ¹⁹⁹Au nanoparticles can be employed to produce a theranostic agent for cancer treatment.

Non-radioactive nanoparticles have been investigated for target specificity and increased retention for significant improvement in the treatment of the prostate and various inoperable tumors. See for example, Raghuraman Kannan et al, “Functionalized radioactive gold nanoparticles in tumor therapy” WIRES Nanomed Nanobiotechnol 2012, 4:42-51, doi: 10.1002/wnaan.161, for validation of the hypothesis that Gum Arabic-functionalized radioactive gold nanoparticles have high affinity toward cancer.

Artisans have also explored methods to produce antibody conjugated nanoparticles. An example is Hainfeld, U.S. Pat. No. 8,033,977. Hainfeld '977 discloses metal core particles, including gold particles, surrounded by a surface or shell layer of another material, such as molecules containing sulfur, phosphorous, amines or molecules with a thiol group. The shell layer can also include proteins, antibodies and fragments, or these can be linked. The technique for fabricating the targeted nanoparticles is during the synthesis of the particles, which can affect the targeting moiety or ligand.

For any given nanoconstruct, Hainfield et. al. does not describe a process to determine a priori the efficacy of the nanoparticle conjugate for treatment of disease. Therefore, there is a need for a process to determine a priori the efficacy of a nanoparticle conjugate comprising a nanoparticle and one or more antibodies of a single type for treatment of disease.

### SUMMARY OF INVENTION

A preferred embodiment is a method of formation of nanoparticle conjugates for cancer diagnostics, imaging and therapy, wherein the method allows determination a priori the efficacy of the nanoparticle conjugate for treatment of disease. A preferred stable pharmaceutical formulation includes a single antibody agent retaining its functionality in accordance with a predetermination of an a priori efficacy and electrostatically or covalently linked to a nanoparticle in a predetermined ratio in accordance with the a priori efficacy.

A second embodiment is a method of formation of nanoparticle conjugate for cancer diagnostics, imaging and therapy, wherein the composite comprises a single antibody type bonded to a nanoparticle via a linker to form a conjugate having either electrostatic or covalent bonding as may be chosen such that the original properties of the single antibody type are most fully retained.

The antibody agent may be human or animal antibody, antibody fragment, affibody, a small molecule, a recombinant humanized monoclonal antibody, or anti-hapten antibody or peptide, cyclic peptide or toxin. The nanoparticle may be a metallic or non-metallic nanoparticle, but is preferably a gold nanoparticle (AuNP). The linker between said antibody (affibody, small molecule, peptide, toxin, fragment, recombinant humanized monoclonal antibody or anti-hapten antibody) and said nanoparticle linked via thiol and ethylene glycol. The exemplary conjugates include electrostatic bonded pertuzumab—AuNP conjugates, covalent bonded pertuzumab—AuNP conjugates, electrostatic bonded trastuzumab—AuNP conjugates, and covalent bonded trastuzumab—AuNP conjugates.

A third embodiment is a method of formation of nanoparticle conjugate for cancer diagnostics, imaging and therapy, wherein the composite comprises a single antibody type bonded to a nanoparticle via a linker to form a conjugate having either electrostatic or covalent bonding as may be chosen such that the original properties of the single antibody type are most fully retained for treatment of disease.

### BRIEF DESCRIPTION OF THE DRAWINGS

FIG. 1 Schematic diagram of preferred process to bond pertuzumab to gold nanoparticle via predetermined selection route for electrostatic attachment or covalent conjugation;

FIGS. 2A and 2B Schematic diagram of preferred purification methods for pertuzumab bonded to gold nanoparticle nanoconjugates by two different techniques for both electrostatic and covalent conjugation; purification by conventional centrifugation (noted by A), and purification using high performance liquid chromatography (HPLC), and isolation by size exclusion chromatography (SEC) (noted by B).

FIG. 3 Comparison of the HPLC chromatograms of experimental nanoconjugates synthesized by both electrostatic and covalent attachment, including an overlay of AuNP-PER conjugate chromatograms prepared by both electrostatic (2B) and covalent (3B) procedures, along with a chromatogram for free monoclonal antibody used for conjugation.

FIG. 4 TEM Images of nanoconjugates 2A, 2B, 3A and 3B obtained by electrostatic adsorption and covalent attachment

FIGS. 5A-5D Histograms respectively showing size distributions obtained from TEM for 2A formed by electrostatic conjugation, for 2B formed by electrostatic conjugation isolated by centrifugation, for 3A formed by covalent conjugation, and for 3B formed by covalent conjugation isolated by size exclusion chromatography (SEC).

FIGS. 6A-6B 6A—Super-imposed hydrodynamic diameter profiles for citrate stabilized gold pegylated AuNPs and for pegylated AuNPs with antibody conjugation formed by electrostatic conjugation (PM) with purification by conventional centrifugation (denoted by 2A). Pegylation was accompanied by an approximate 10 nm increase in hydrodynamic diameter when compared with non-pegylated AuNPs. 6B-Super-imposed hydrodynamic diameter profiles for citrate stabilized gold pegylated AuNPs and for pegylated AuNPs with antibody conjugation formed by electrostatic conjugation (PM) with purification by HPLC and isolation by SEC (denoted by 2B). Pegylation was accompanied by an approximate 10 nm increase in hydrodynamic diameter when compared with non-pegylated AuNPs.

FIGS. 7A-7B 7A-Super-imposed hydrodynamic diameter profiles for citrate stabilized gold pegylated AuNPs and for pegylated AuNPs with antibody conjugation formed by covalent conjugation (COV) with purification by conventional centrifugation (denoted by 3A). Pegylation was accompanied by an approximate 10 nm increase in hydrodynamic diameter when compared with non-pegylated AuNPs. 7B-Super-imposed hydrodynamic diameter profiles for citrate stabilized gold pegylated AuNPs and for pegylated AuNPs with antibody conjugation formed by covalent conjugation (COV) with purification by HPLC and isolation by SEC (denoted by 3B).

FIG. 8 Super-imposed hydrodynamic diameter profiles for citrate stabilized (non-pegylated) AuNPs, for citrate stabilized pegylated AuNPs, and for pegylated AuNPs with antibody conjugation formed by covalent conjugation (COV) with purification by HPLC and isolation by SEC (denoted by 3B). Pegylation was accompanied by an approximate 10 nm increase in hydrodynamic diameter when compared with non-pegylated AuNPs. The increased diameter of approximately 24 nm (over non-pegylated AuNPs) for nanoconjugate 3B suggests presence of a physioadsorbed antibody in the covalently attached HPLC purified and SEC isolated nanoconjugate.

FIGS. 9A-9B 9A—measured values and summary statements of several observed changes for six nanoconjugates that were formed and purified by different procedures, as identified. 9B—Measured values include locations of plasmon absorption peak in UV-Vis spectra, hydrodynamic diameters, and zeta potentials for each nanoconjugate.

FIGS. 10A-10B Schematic diagrams illustrating the nanoconjugate for which protein quantification was measured, including electrostatic bonded constructs purified by two different means; namely, centrifugation and purification by HPLC and isolation by SEC, and covalent bonded constructs also purified by two different means; namely, by centrifugation and by HPLC purification and SEC isolation.

FIGS. 11-11B Measured values for protein present in the four identified nanoconjugates, prepared by either electrostatic or covalent methods and purified by either centrifugal or HPLC methods.

FIG. 12 Schematic diagram of a preferred method for proteomic analyses of nanoconjugates formed by the electrostatic method and by the covalent method.

FIGS. 13A-13B Histograms showing spectral count, average spectral count, % sequence coverage, and % average sequence coverage for heavy and light chains for several gold nanoparticle—pertuzumab nanoconjugates, including those formed by the electrostatic method and by the covalent method using centrifuge isolation.

FIGS. 14A-14B Histograms showing spectral count, average spectral count, % sequence coverage, and % average sequence coverage for heavy and light chains for several gold nanoparticle—pertuzumab nanoconjugates, including those formed by the electrostatic method and by the covalent method using HPLC isolation.

FIG. 15 Sequence coverage results from proteomics analysis, for both heavy and light chains of pertuzumab conjugated to gold nanoparticles with statistical significance Fisher's exact test.

FIG. 16 Sequence coverage results from proteomics analysis, for both heavy and light chains of pertuzumab conjugated to gold nanoparticles with statistical significance t-test.

FIG. 17 Sequence coverage results from proteomics analysis, for both heavy and light chains of pertuzumab conjugated via electrostatic attachment to gold nanoparticles.

FIG. 18 Sequence coverage results from proteomics analysis, for both heavy and light chains of pertuzumab conjugated via covalent attachment to gold nanoparticles.

FIGS. 19A-19B Comparison of spectral count, average spectral count, % sequence coverage, and % average sequence coverage for heavy and light chains, for centrifuged and for purified by HPLC and isolated by SEC nanoconjugates prepared by electrostatic (PM) conjugation method.

FIGS. 20A-20B Comparison of spectral count, average spectral count, % sequence coverage, and % sequence coverage for heavy and light chains, for centrifuged and for HPLC purified and SEC isolated nanoconjugates prepared by covalent conjugation method.

FIG. 21 Comparison of spectral counts and sequence coverage obtained from proteomics data for nanoconjugates prepared by electrostatic (PM) and covalent conjugation methods.

FIGS. 22A-22B Comparison of binding affinity data as determined by direct ELISA procedure for pertuzumab conjugated to gold nanoparticles isolated by centrifugation and HPLC methods, for nanoconjugates prepared by electrostatic (PM) and covalent conjugation methods.

FIGS. 23A-23B Comparison of binding affinity data as determined by direct ELISA procedure for pertuzumab conjugated to gold nanoparticles isolated by centrifugation and HPLC methods, obtained by sandwich ELISA, for nanoconjugates prepared by electrostatic (PM) and covalent conjugation methods.

FIG. 24 TEM images obtained from immuno histochemistry studies that validate the binding affinity of 3B antibody pertuzumab AuNP nanoconjugates purified by HPLC isolated by SEC procedure (large 15 nm dark spots) with anti-human IgG AuNP nanoconjugate (smaller 5 nm light grey spots). The upper panel illustrates attraction of these two entities. The final separation distance measured from an AuNP surface (through a PEG linker) to an anti-human AuNP IgG was approximately 16-18 nm.

FIGS. 25A-25D Dark field microscope images for covalently attached AuNP pertuzumab nanoconjugates (FIGS. 25A-B) and electrostatically attached AuNP pertuzumab nanoconjugates (FIGS. 25C-D) that demonstrate their binding affinity to HER2 positive SkBr3 breast cancer cells. The covalently attached gold-antibody nanoconjugate showed higher uptake and greater internalization through effective HER2 binding than did the physioadsorbed gold-antibody nanoconjugate that was formed by electrostatic attachment, with the latter showing minimal or no internalization.

FIG. 26 Cellular uptake, as measured in Au content in μg using ICP analysis, for SKBR-3 cells with over expressed HER2 receptors when exposed to (pertuzumab) antibody—gold nanoparticle nanoconjugates for 24 hours, where the nanoconjugates were formed and isolated by four different methods: electrostatic attachment and centrifuge separation (PM-PELLET), electrostatic attachment and HPLC purification and SEC isolation (PM-HPLC), covalent attachment and centrifuge separation (COV-PELLET), covalent attachment and HPLC purification and SEC isolation (COV-HPLC).

FIG. 27 TEM imaging studies illustrating cellular uptake for electrostatically formed nanoconjugates (2B). A large fraction of electrostatically formed conjugated nanoconjugates were found to be located in lysosomes and late endosomes, whereas the amount located in cytoplasm was minimal.

FIG. 28 TEM imaging studies illustrating cellular uptake for covalently formed nanoconjugates (3B). A large fraction of covalently formed conjugated nanoconjugates were observed as aggregates located in cytoplasm.

FIG. 29 Schematic showing steps in preferred syntheses of trastuzumab gold antibody nanoconjugates. Syntheses were performed following two different procedures; namely, by electrostatic attachment (schematically represented in left branch), and by covalent conjugation (schematically represented in right branch).

FIG. 30 Comparison of the HPLC chromatograms of trastuzumab nanoconjugates synthesized by both electrostatic and covalent attachment. Shown is an overlay of AuNP-PER conjugate chromatograms prepared by both electrostatic (2H) and covalent (3H) procedures, along with a chromatogram for free monoclonal antibody used for conjugation.

FIGS. 31A-31B Measured values and summary statements of several observed changes for six nanoconjugates that were formed and purified by different procedures, as identified. Measured values include locations of plasmon absorption peak in UV-Vis spectra, hydrodynamic diameters, and zeta potentials for each trastuzumab nanoconjugate.

FIG. 32 Super-imposed hydrodynamic diameter profiles for citrate stabilized (non-pegylated) AuNPs, for citrate stabilized pegylated AuNPs, and for pegylated AuNPs with antibody conjugation formed by covalent conjugation (COV) with purification by HPLC and isolated by SEC (denoted by 2H,3H). Pegylation was accompanied by an approximate 10 nm increase in hydrodynamic diameter when compared with non-pegylated AuNPs. The increased diameter of approximately 24 nm (over non-pegylated AuNPs) for nanoconjugate 3H suggests presence of a physioadsorbed antibody in the covalently attached purified by HPLC and isolated by SEC nanoconjugate.

FIG. 33A-33B Measured values for protein present in the four identified trastuzumab conjugates, prepared by either electrostatic or covalent methods and purified by either centrifugal or HPLC purified and SES isolatedmethods, are listed.

FIG. 34 Schematic illustrating the process followed for proteomic analyses of trastuzumab nanoconjugates formed by the electrostatic method (2H) and by the covalent method (3H).

FIG. 35 Histograms showing spectral count, average spectral count, % sequence coverage, and % average sequence coverage for heavy and light chains for several gold nanoparticle—trastuzumab nanoconjugates, including those formed by the electrostatic method and by the covalent method using centrifuge isolation.

FIG. 36 Spectral counts and sequence coverage for two methods of conjugation (electrostatic and covalent), and for each of the two conjugation methods are listed the values for spectral counts and sequence coverage for heavy chains and for light chains.

FIG. 37 Spectral count results from proteomics analysis, for both heavy and light chains of trastuzumab gold nanoconjugates obtained by electrostatic and covalent conjugation procedure.

FIG. 38 Sequence coverage results from proteomics analysis, for both heavy and light chains of trastuzumab gold nanoconjugates obtained by electrostatic conjugation.

FIG. 39 Sequence coverage results from proteomics analysis, for both heavy and light chains of trastuzumab gold nanoconjugates obtained by covalent conjugation.

FIGS. 40-41 Experiments to optimize ratio of antibodies (of single type) to gold nanoparticles.

FIG. 42 Experiment to achieve favorable antibody agent orientation via linker weight.

FIG. 43 Experiment with different weight of linker showing change from 2000 to 3400 Da.

FIG. 44 ELISA binding plots of experimental nanoconjugates.

FIG. 45 In vitro cytotoxicity studies in breast cancer SkBR3 cells when pertuzumab gold nanoconjugates obtained by electrostatic and covalent conjugation were incubated for 96 hrs and analysed by MTT assay.

FIG. 46 In vitro cytotoxicity studies in breast cancer SkBR3 cells when trastuzumab gold nanoconjugates obtained by electrostatic and covalent conjugation were incubated for 96 hrs and analysed by MTT assay.

FIG. 47 Comparison of in vitro cytotoxicity studies in breast cancer SkBR3 and MCF7 cells when trastuzumab and pertuzumab gold nanoconjugates obtained by electrostatic and covalent conjugation were incubated for 96 hrs and analysed by MTT assay.

FIG. 48 Western blot analysis of pertuzumab gold nanoconjugates obtained by electrostatic and covalent conjugation in breast cancer SkBR3 cells analyzing the expression of proteins, HER2, phosphor-HER2, HER3, phospho-HER3, Akt, phospho-Akt, ERK, phospho-ERK, MEK, phospho-MEK, and actin.

FIG. 49 Western blot analysis of pertuzumab gold nanoconjugates obtained by electrostatic and covalent conjugation in breast cancer SkBR3 cells analyzing the expression of proteins, HER2, phospho-HER2.

FIG. 50 Western blot analysis of pertuzumab gold nanoconjugates obtained by electrostatic and covalent conjugation in breast cancer SkBR3 cells analyzing the expression of proteins, HER3, phospho-HER3.

FIG. 51 Western blot analysis of pertuzumab gold nanoconjugates obtained by electrostatic and covalent conjugation in breast cancer SkBR3 cells analyzing the expression of proteins, AkT, phospho-AkT.

FIG. 52 Western blot analysis of pertuzumab gold nanoconjugates obtained by electrostatic and covalent conjugation in breast cancer SkBR3 cells analyzing the expression of proteins, ERK1, ERK2, phospho-ERK.

FIG. 53 Western blot analysis of pertuzumab gold nanoconjugates obtained by electrostatic and covalent conjugation in breast cancer SkBR3 cells analyzing the expression of proteins, MEK, and phospho-MEK.

FIG. 54 Western blot analysis of pertuzumab gold nanoconjugates obtained by electrostatic and covalent conjugation in breast cancer SkBR3 cells analyzing the expression of proteins, Actin.

FIG. 55 Summary of western blot analysis of pertuzumab gold nanoconjugates obtained by electrostatic and covalent conjugation in breast cancer SkBR3 cells analyzing the expression of proteins, HER2, phosphor-HER2, HER3, phospho-HER3, Akt, phospho-Akt, ERK, phospho-ERK, MEK, phospho-MEK, and actin.

### DETAILED DESCRIPTION OF THE INVENTION

A preferred embodiment is a method of formation of nanoparticle conjugates for cancer diagnostics, imaging and therapy, wherein the method allows determination a priori the efficacy of the nanoparticle conjugate for treatment of disease. A method of formation of nanoparticle conjugate for cancer diagnostics, imaging and therapy, wherein the composite includes a single antibody type bonded to a nanoparticle via a linker to form a conjugate having either electrostatic or covalent bonding as may be chosen such that the original properties of the single antibody type are most fully retained.

Preferred embodiments provide methods for formation of nanoparticle conjugates comprising a biocompatible single antibody agent (single type of antibody agent, i.e. one or more antibody agents of a single type) bonded to a nanoparticle via a linker. The antibody retains its functionality and the nanconjugate can act as a therapeutic agent for the treatment of cancer, such as breast cancer. In preferred embodiments, single human antibody type bonded to ¹⁹⁸Au nanoparticles can serve as imaging agents, thereby acting as theranostic agents for imaging, therapy and diagnostic purposes.

Experiments show that Gum Arabic-functionalized radioactive and non-radioactive gold nanoparticles and human antibody agents, such as pertuzumab and trastuzumab, possess an affinity towards cancer. Non-metallic nanoparticles can be used in additional embodiments. Such non-metallic nanoparticles can be polymeric and or lipid or miscelles in which antibody, affibody, peptide, cyclic peptide or small molecules, toxins, fragments are attached via a similar linker will exhibit property identical to those attached to metallic nanoparticles after conjugation

Both Gum Arabic-functionalized radioactive gold nanoparticles and non-radioactive gold nanoparticles possess an affinity towards cancer due to the affinity of Gum Arabic to cancer. Gold nanoparticles can be fabricated in the range of about 3 to 80 nm, and preferably in the range of 5 to 15 nm in preferred nanoconjugates.

A preferred linker is thiol or ethylene glycol, e.g. monoethylene glycol, diethylene glycol, and polyethylene glycol. The thiol is preferably at least one of the thioctic acid, monothioctic acid, dithioctic acid, and trithioctic acid. A formulation is suspended in an aqueous solution and is is sterile and can be for intravenous (IV), subcutaneous (SQ), intraperitoneal (IP), intratumoral (IT) or intramuscular (IM) administration.

Gum Arabic and human antibody agents, such as pertuzumab and trastuzumab, have an affinity towards cancer, which should provide affinity to cancer for gold nanoparticles functionalized with Gum Arabic and gold nanoparticles functionalized with human antibody agents, such as pertuzumab and trastuzumab. Furthermore, the affinity for cancer for gold nanoparticles functionalized with human antibody agents, such as pertuzumab and trastuzumab, is expected to be independent of whether the gold nanoparticles were either non-radioactive or radioactive. The affinity for human cancer for gold nanoparticles functionalized with human antibody agents, such as pertuzumab and trastuzumab, is expected to be independent of whether the coating of the gold nanoparticles during synthesis was Gum Arabic, citrate or another suitable coating entity since the affinity to cancer possessed by gold nanoparticles functionalized with human antibody agents arises from the presence and properties of the human antibody agent bonded to the gold nanoparticle—whether non-radioactive or radioactive.

Experimental human antibody-AuNP conjugates formed in accordance with preferred embodiments of invention were synthesized using non-radioactive 197Au.

The nature of the binding to cancer cells of human antibody-AuNP conjugates indicates that nanoconjugates containing any and all admixture ratios of non-radioactive 197Au, radioactive 198Au, and radioactive 199Au are expected to the same in pharmaceutical applications.

Preferred embodiments include single human antibody—gold nanoparticle conjugates wherein the gold nanoparticle comprised of the element 197Au may be incorporated into a suitable pharmaceutical formulation wherein the gold nanoparticles are coated with citrate, and used in medical applications such as diagnosis, imaging, and therapy of cancers, including, but not limited to, human cancers, and wherein the therapy is by use of one or more of the techniques in the list including, but not limited to, chemotherapy, brachy therapy, and other techniques, and wherein the imaging is by use of one or more of the techniques in the list including, but not limited to, MRI, CAT, ultrasound and other techniques.

On the basis of the results of this invention and the similar chemical behavior and binding affinity to human cancer possessed by human antibodies and human antibody fragments and modified human antibodies, affibodies, peptides, cyclic peptides and toxins, the experiments support nanoconjugates of the invention that include human antibody fragments and modified human antibodies—gold nanoparticle conjugates (including non-radioactive and radioactive gold elements), for use in a suitable pharmaceutical formulation for use in medical applications such as diagnosis, imaging, and therapy of human cancers.

In a preferred embodiment, a linker for a nanoparticle conjugate of gold nanoparticle and a pertuzumab antibody possesses a molecular weight of 3400 Da.

In a preferred embodiment, a linker for a nanoparticle conjugate of gold nanoparticle and a pertuzumab antibody possesses a molecular weight of 2000 Da.

In a preferred embodiment, a linker for a nanoparticle conjugate of gold nanoparticle and a trastuzumab antibody possesses a molecular weight of 3400 Da.

In a preferred embodiment, a linker for a nanoparticle conjugate of gold nanoparticle and a trastuzumab antibody possesses a molecular weight of 2000 Da.

Antibodies in nanoconjugates of the invention can take various forms. For instance, they may be native antibodies, as naturally found in mammals. Native antibodies are made up of heavy chains and light chains. The heavy and light chains are both divided into variable domains and constant domains. The ability of different antibodies to recognize different antigens arises from differences in their variable domains, in both the light and heavy chains. Light chains of native antibodies in vertebrate species are either kappa (.kappa.) or lambda (.lambda.), based on the amino acid sequences of their constant domains. The constant domain of a native antibody's heavy chains will be .alpha., .delta., .epsilon., .gamma. or .mu., giving rise respectively to antibodies of IgA, IgD, IgE, IgG, or IgM class. Classes may be further divided into subclasses or isotypes e.g. IgG1, IgG2, IgG3, IgG4, IgA, IgA2, etc. Antibodies may also be classified by allotype e.g. a .gamma. heavy chain may have G1m allotype a, f, x or z, G2m allotype n, or G3m allotype b0, b1, b3, b4, b5, c3, c5, g1, g5, s, t, u, or v; a .kappa. light chain may have a Km(1), Km(2) or Km(3) allotype. A native IgG antibody has two identical light chains (one constant domain C.sub.L and one variable domain V.sub.L) and two identical heavy chains (three constant domains C.sub.H1 C.sub.H2 & C.sub.H3 and one variable domain V.sub.H), held together by disulfide bridges. The domain and three-dimensional structures of the different classes of native antibodies are well known.

Where an antibody in nanoconjugates of the invention has a light chain with a constant domain, it may be a .kappa. or .lambda. light chain (although, in some embodiments, antibodies must have a .lambda. light chain). Where an antibody of the invention has a heavy chain with a constant domain, it may be a .alpha., .delta., .epsilon., .gamma. or .mu. heavy chain. Heavy chains in the .gamma. class (i.e. IgG antibodies) are preferred. Antibodies of the invention may have any suitable allotype (see above).

Antibodies in nanoconjugates of the invention may be fragments of native antibodies that retain antigen binding activity. For instance, papain digestion of native antibodies produces two identical antigen-binding fragments, called “Fab” fragments, each with a single antigen-binding site, and a residual “Fc” fragment without antigen-binding activity. Pepsin treatment yields a “F(ab′).sub.2” fragment that has two antigen-binding sites. “Fv” is the minimum fragment of a native antibody that contains a complete antigen-binding site, consisting of a dimer of one heavy chain and one light chain variable domain. Thus an antibody of the invention may be Fab, Fab′, F(ab′).sub.2, Fv, or any other type, of fragment of a native antibody. Antibodies of the invention may incorporate Dual-Affinity Re-Targeting (DART) platform technology, which is focused on dual specificity “antibody-like” therapeutic proteins capable of targeting multiple different epitopes with a single recombinant molecule.

An antibody in nanoconjugates of the invention may be a “single-chain Fv” (“scFv” or “sFv”), comprising a V.sub.H and V.sub.L domain as a single polypeptide chain. Typically the V.sub.H and V.sub.L domains are joined by a short polypeptide linker (e.g. .gtoreq.12 amino acids) between the V.sub.H and V.sub.L domains that enables the scFv to form the desired structure for antigen binding. A typical way of expressing scFv proteins, at least for initial selection, is in the context of a phage display library or other combinatorial library. Multiple scFvs can be linked in a single polypeptide chain.

An antibody in nanoconjugates of the invention may be a “diabody” or “triabody” etc., comprising multiple linked Fv (scFv) fragments. By using a linker between the V.sub.H and V.sub.L domains that is too short to allow them to pair with each other (e.g. <12 amino acids), they are forced instead to pair with the complementary domains of another Fv fragment and thus create two antigen-binding sites.

An antibody in nanoconjugates of the invention may be a single variable domain or VHH antibody. Antibodies naturally found in camelids (e.g. camels and llamas) and in sharks contain a heavy chain but no light chain. Thus antigen recognition is determined by a single variable domain, unlike a mammalian native antibody. The constant domain of such antibodies can be omitted while retaining antigen-binding activity. One way of expressing single variable domain antibodies, at least for initial selection, is in the context of a phage display library or other combinatorial library.

An antibody in nanoconjugates of the invention may be a “domain antibody” (dAb). Such dAbs are based on the variable domains of either a heavy or light chain of a human antibody and have a molecular weight of approximately 13 kDa (less than one-tenth the size of a full antibody). By pairing heavy and light chain dAbs that recognize different targets, antibodies with dual specificity can be made, dAbs are cleared from the body quickly, but can be sustained in circulation by fusion to a second dAb that binds to a blood protein (e.g. to serum albumin), by conjugation to polymers (e.g. to a polyethylene glycol), or by other techniques.

An antibody in nanoconjugates of the invention may be a chimeric antibody, having constant domains from one organism (e.g. a human) but variable domains from a different organism (e.g. non-human). Chimerization of antibodies was originally developed in order to facilitate the transfer of antigen specificity from easily-obtained murine monoclonal antibodies into a human antibody, thus avoiding the difficulties of directly generating human monoclonal antibodies. Because the inventor already provided human antibodies as a starting point for further work then chimerization will not typically be required for performing the invention. If non-human antibodies are generated, however, then they can be used to prepare chimeric antibodies. Similarly, if human antibodies of the invention are to be used in non-human organisms then their variable domains could be joined to constant domains from the non-human organism.

An antibody in nanoconjugates of the invention may be a CDR-grafted antibody. With human antibodies as a starting point then, as for chimerisation, CDR grafting will not typically be required, but such an antibody can also be used.

Thus the term “antibody” as used herein encompasses a range of proteins having diverse structural features (usually including at least one immunoglobulin domain having an all-.beta. protein fold with a 2-layer sandwich of anti-parallel .beta.-strands arranged in two .beta.-sheets), but all of the proteins possess the ability to bind to proteins.

Antibodies in nanoconjugates of the invention may include a single antigen-binding site (e.g. as in a Fab fragment or a scFv) or multiple antigen-binding sites (e.g. as in a F(ab′).sub.2 fragment or a diabody or a native antibody). Where an antibody has more than one antigen-binding site then advantageously it can result in cross-linking of antigens.

Where an antibody has more than one antigen-binding site, the antibody may be mono-specific (i.e. all antigen-binding sites recognize the same antigen) or it may be multi-specific (i.e. the antigen-binding sites recognize more than one antigen). Thus, in a multi-specific antibody, at least one antigen-binding site will recognize a H5N1 influenza A virus and at least one antigen-binding site will recognize a different antigen.

An antibody in nanoconjugates of the invention may include a non-protein substance e.g. via covalent conjugation. For example, an antibody may include a radio-isotope e.g. the Zevalin™ and Bexxar™ products include .sup.90Y and .sup.131I isotopes, respectively. As a further example, an antibody may include a cytotoxic molecule e.g. Mylotarg™ is linked to N-acetyl-.gamma.-calicheamicin, a bacterial toxin. As a further example, an antibody may include a covalently-attached polymer e.g. attachment of polyoxyethylated polyols or polyethylene glycol (PEG) has been reported to increase the circulating half-life of antibodies.

An antibody in nanoconjugates of the invention may include a peptide, cyclic peptide, affibody or toxin.

In some embodiments of the invention, an antibody can include one or more constant domains (e.g. including C.sub.H or C.sub.L domains). As mentioned above, the constant domains may form a .kappa. or .lambda. light chain or an .alpha., .delta., .epsilon., .gamma. or .mu. heavy chain. Where an antibody of the invention includes a constant domain, it may be a native constant domain or a modified constant domain. A heavy chain may include either three (as in .alpha., .gamma., .delta. classes) or four (as in .mu., .epsilon. classes) constant domains. Constant domains are not involved directly in the binding interaction between an antibody and an antigen, but they can provide various effector functions, including but not limited to: participation of the antibody in antibody-dependent cellular cytotoxicity (ADCC); C1 q binding; complement dependent cytotoxicity; Fc receptor binding; phagocytosis; and down-regulation of cell surface receptors.

The constant domains can form a “Fc region”, which is the C-terminal region of a native antibody's heavy chain. Where an antibody of the invention includes a Fc region, it may be a native Fc region or a modified Fc region. A Fc region is important for some antibodies' functions e.g. the activity of Herceptin™ is Fc-dependent. Although the boundaries of the Fc region of a native antibody may vary, the human IgG heavy chain Fc region is usually defined to stretch from an amino acid residue at position Cys226 or Pro230 to the heavy chain's C-terminus. The Fc region will typically be able to bind one or more Fc receptors, such as a Fc.gamma.RI (CD64), a Fc.gamma.RII (e.g. Fc.gamma.RIIA, Fc.gamma.RIIB1, Fc.gamma.RIIB2, Fc.gamma.RIIC), a Fc.gamma.RIII (e.g. Fc.gamma.RIIIA, Fc.gamma.RIIIB), a FcRn, Fc.alpha.R (CD89), Fc.delta.R, Fc.mu.R, a Fc.epsilon.RI (e.g. Fc.epsilon.RI.alpha..beta..gamma..sub0.2 or Fc.epsilon.RI.alpha..gamma..sub0.2), Fc.epsilon.RII (e.g. Fc.epsilon.RIIA or Fc.epsilon.RIIB), etc. The Fc region may also or alternatively be able to bind to a complement protein, such as C1q. Modifications to an antibody's Fc region can be used to change its effector function(s) e.g. to increase or decrease receptor binding affinity. For instance, reference 39 reports that effector functions may be modified by mutating Fc region residues 234, 235, 236, 237, 297, 318, 320 and/or 322. Similarly, reference 40 reports that effector functions of a human IgG1 can be improved by mutating Fc region residues (EU Index Kabat numbering) 238, 239, 248, 249, 252, 254, 255, 256, 258, 265, 267, 268, 269, 270, 272, 276, 278, 280, 283, 285, 286, 289, 290, 292, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 and/or 439. Modification of Fc residues 322, 329 and/or 331 is reported in reference 41 for modifying C1q affinity of human IgG antibodies, and residues 270, 322, 326, 327, 329, 331, 333 and/or 334 are selected for modification in reference 42. Mapping of residues important for human IgG binding to FcRI, FcRII, FcRIII, and FcRn receptors is reported in reference 43, together with the design of variants with improved FcR-binding properties. Whole C.sub.H domains can be substituted between isotypes e.g. reference 44 discloses antibodies in which the C.sub.H3 domain (and optionally the C.sub.H2 domain) of human IgG4 is substituted by the C.sub.H3 domain of human IgG1 to provide suppressed aggregate formation. It has been reported that mutation of arginine at position 409 (EU index Kabat) of human IgG4 to e.g. lysine shows suppressed aggregate formation. Mutation of the Fc region of available monoclonal antibodies to vary their effector functions is known e.g. known mutation studies for RITUXAN™ to change C1q-binding, and known mutation studies for NUMAX™ to change FcR-binding, with mutation of residues 252, 254 and 256 giving a 10-fold increase in FcRn-binding without affecting antigen-binding.

Antibodies in nanoconjugates of the invention will be glycosylated in preferred embodiments. N-linked glycans attached to the C.sub.H2 domain of a heavy chain, for instance, can influence C1q and FcR binding, with aglycosylated antibodies having lower affinity for these receptors. The glycan structure can also affect activity e.g. differences in complement-mediated cell death may be seen depending on the number of galactose sugars (0, 1 or 2) at the terminus of a glycan's biantennary chain. An antibody's glycans preferably do not lead to a human immunogenic response after administration.

Antibodies in nanoconjugates of the invention can be prepared in a form free from products with which they would naturally be associated. Contaminant components of an antibody's natural environment include materials such as enzymes, hormones, or other host cell proteins.

Antibodies in nanoconjugates of the invention can be used directly (e.g. as the active ingredient for pharmaceuticals or diagnostic reagents), or they can be used as the basis for further development work. For instance, an antibody may be subjected to sequence alterations or chemical modifications in order to improve a desired characteristic e.g. binding affinity or avidity, pharmacokinetic properties (such as in vivo half-life), etc. Techniques for modifying antibodies in this way are known in the art. For instance, an antibody may be subjected to “affinity maturation”, in which one or more residues (usually in a CDR) is mutated to improve its affinity for a target antigen. Random or directed mutagenesis can be used, but reference 47 describes affinity maturation by V.sub.H and V.sub.L domain shuffling as an alternative to random point mutation. Published reports show how NUMAX™ was derived by a process of in vitro affinity maturation of the CDRs of the heavy and light chains of SYNAGIS™, giving an antibody with enhanced potency and 70-fold greater binding affinity for RSV F protein.

Preferred antibodies in nanoconjugates of the invention are specific for Her2. Thus the antibody will have a tighter binding affinity for that antigen than for an arbitrary control antigen e.g. than for a human protein. Preferred antibodies have nanomolar or picomolar affinity constants for target antigens e.g. 10.sup.-9 M, 10.sup.-10 M, 10.sup.-11 M, 10.sup.-12 M, 10.sup.-13 M or tighter). Such affinities can be determined using conventional analytical techniques e.g. using surface plasmon resonance techniques as embodied in BIAcore™ instrumentation and operated according to the manufacturer's instructions. Radio-immunoassay using radiolabeled target antigen (Her2) is another method by which binding affinity may be measured.

For the present invention, the inventors believe, without limiting to the invention, the human antibody bonded to the AuNP may be an antibody type in the list including, but not limited to, an antibody, an antibody fragment, an affibody, a peptide, a cyclic peptide, a toxin, a small molecule, a recombinant humanized monoclonal antibody, a rabbit antibody, a goat antibody, a mouse antibody, and an anti-hapten antibody.

For the present invention, the inventors believe, without limiting to the invention, the human antibody bonded to the AuNP may be a human antibody in the list comprising, but not limit to, all human antibodies contained in U.S. Pat. No. 8,124,092, filed Mar. 3, 2008, that have application for diagnosis, imaging and therapy for human cancer. including, but not limit to, all human antibody variants, FABS, bi-specifics, and human antibodies with applications for dual affinity retargeting therapy (DART), that have application for diagnosis, imaging and therapy for human cancer. For the present invention, the inventors believe, without limiting to the invention, the human antibody bonded to the AuNP may be a human antibody in the list comprising, but not limit to, all human antibodies now provided that have application for diagnosis, imaging and therapy for human cancer.

Specific radioactive emitting and non-emitting gold nanoparticles have individually acted as imaging and therapy agents. Specific human antibodies have individually acted as therapy agents. However, singular agents with dual imaging and therapeutic (“theranostic”) capabilities that includes a human antibody bonded to a gold nanoparticle have not been provided to the inventors' knowledge. The present inventors have recognized that a dual theranostic agent would provide tremendous consistency in the follow-up of therapy studies and could also minimize regulatory steps leading to final approval by the Food and Drug Administration (FDA). The inventors have also recognized that theranostic properties of a human antibody bonded to a gold nanoparticle offer realistic clinical possibilities for use as dual imaging, diagnostic and therapy agents.

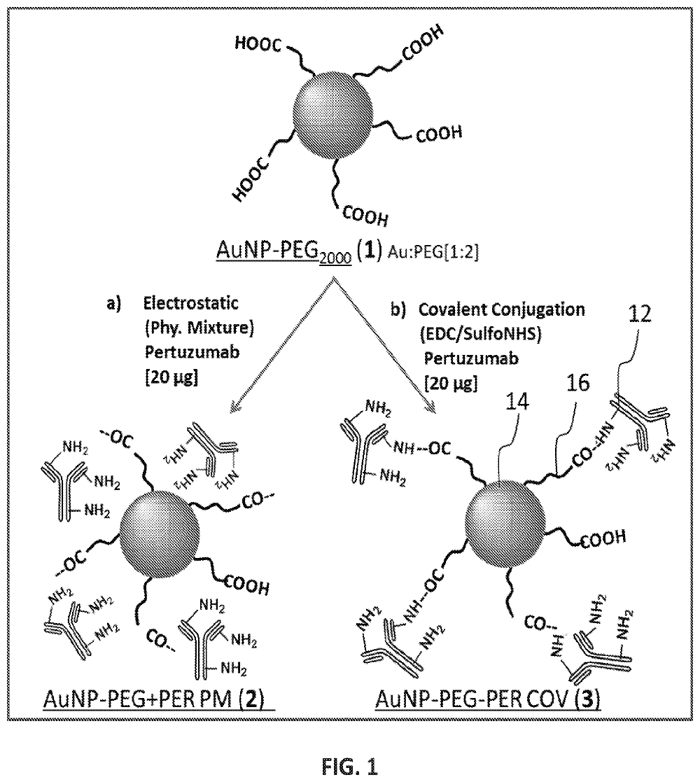

Conjugates comprising single human antibody type bonded to gold nanoparticles can be synthesized under clinical settings. FIG. 1 illustrates preferred processes for covalent (COV) and electrostatic (PM) synthesis. A single type of agent 12 is conjugated to a nanoparticle 14 via a linker (COOH) 16.

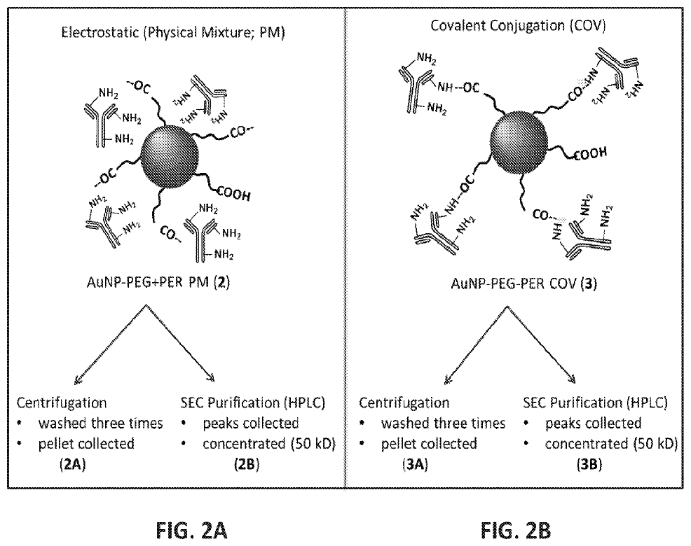

Consistent with FIG. 1, citrate coated gold nanoparticles were made and human antibody pertuzumab was bonded to the gold nanoparticle by two different bonding methods; namely, by electrostatic bonding and by covalent bonding. Tools based on proteomics were used to characterize the pertuzumab—gold nanoparticle conjugate with a PEG linker, herein labeled as AuNP-PEG-PER. FIGS. 2A-2B illustrate purification methods.

The AuNP-PEG conjugate with pertuzumab formed by electrostatic bonding method, i.e., physical bonding method is denoted as AuNP-PEG+PEM PM. The “+” sign located between “PEG” and “PEM” indicates electrostatic bonding (or, physical bonding; hence, PM). “ELE” is sometimes utilized to indicate electrostatic bonding. The AuNP-PER conjugate formed by covalent bonding method is denoted as AuNP-PEG-PER COV. The “−” sign located between “PEG” and “PEM” indicates covalent bonding. Other tags may be placed on either of the names for these two conjugates to indicate method of purification and separation; for example, high pressure liquid chromatography (HPLC) for purification and size exclusion chromatography (SEC) for isolation and PELLET for use of product from centrifugation (for separation), as seen in FIGS. 2A and 2B.

### Experimental Results

Antibody—AuNP Conjugate Synthesis

Citrate stabilized gold nanoparticles were prepared by a modified Frens and Turkevich procedure to ensure complete saturation of AuNPs to yield particles of uniform size and shape (ACS NANO 2013, 7, 1129).

The citrate ions were replaced by direct chemisorption of bifunctional polyethylene glycol (PEG) linker that contained thiol and end terminal carboxyl functionality. The carboxyl terminal was utilized to covalently conjugate antibody. Pegylation of AuNP was performed using both 3400 and 2000 Dalton (Da) linkers, but the antibody conjugates prepared with PEG-2000 showed best results for target binding.

The pegylation was confirmed by incubating AuNP-(PEG-2000)-COOH (1) in 10% NaCl solution and by UV-Vis monitoring of the plasmon absorption of nanoparticles. The pegylated conjugate showed no visible aggregation and no change in surface plasmon resonance peaks, thereby indicating stability of the conjugate.

Antibody conjugation for pertuzumab to AuNP-PEG and for trastuzumab to AuNP-PEG was performed by utilizing two different procedures; namely, by covalent conjugation and by electrostatic attachment, consistent with FIG. 1.

In covalent conjugation, the end carboxyl groups were activated using 1-Ethyl-3-[3-dimethylaminopropyl] carbodiimide hydrochloride/N-hydroxysulfosuccinimide ((EDC)/(Sulfo NHS)) procedure in 4-morpholinoethanesulfonic acid (MES) buffer (pH=4.5) followed by addition of 20 μg of antibody, while in electrostatic attachment, the conjugation was achieved by simple mixing of antibody to AuNP-PEG solution in the absence an activating agent.

Purification by HPLC and Isolation by SEC

The gold antibody nanoconjugates were purified using two different techniques; namely, by (i) conventional centrifugation procedure, and (ii) HPLC purification and SEC isolation. FIGS. 2A-2B.

Antibody-gold conjugates were, in general, purified by centrifugation irrespective of method of preparation (i.e., by either covalent or electrostatic attachment) and have been utilized for both in vitro and in vivo studies.

However, it is always difficult to isolate pure nanoconjugates by centrifugation technique since the excess of protein and other additives remain adsorbed on the surface of AuNPs due to their ionic interactions and notorious sticky nature, even if attached covalently. Therefore, we have utilized an alternative size exclusion chromatography using HPLC to purify and SEC to isolate nanoconjugates.

In size exclusion chromatography, separation is achieved based on the size/molecular weight. Therefore, we anticipated that the separation of excess protein from the antibody gold nanoconjugate could be achieved.

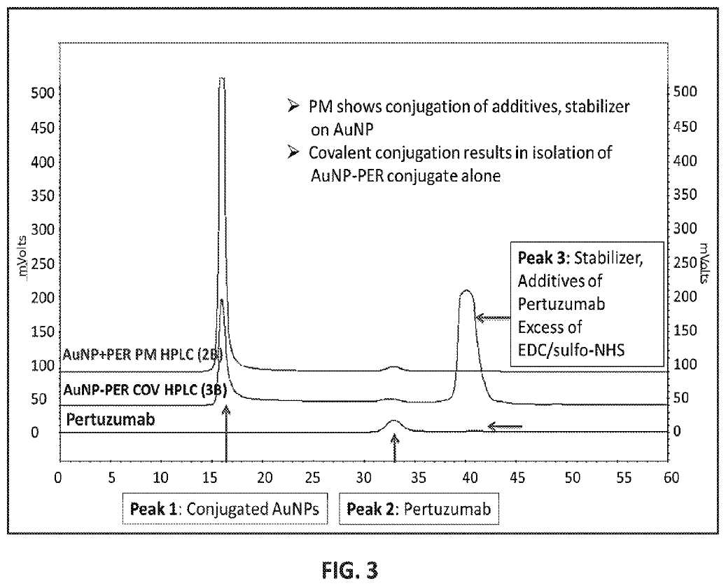

The HPLC chromatograms of nanoconjugates synthesized by both electrostatic and covalent attachment were compared in FIG. 3. An overlay of human antibody AuNP-PEG conjugate prepared for each of pertuzumab conjugate and trastuzumab conjugates, by both electrostatic (2B) and covalent (3B) procedures, along with free monoclonal antibody used for conjugation was prepared. For pertuzumab, free pertuzumab (PER) (˜150 kDa) showed a 33 minute (min) retention time. Additives present in the antibody appeared at 42 min. For pertuzumab, upon injection of the crude antibody-gold nanoconjugate in the SEC column, obtained by both electrostatic and covalent attachment procedures, three different peaks were observed—corresponding to the pertuzumab—AuNP-PEG conjugate (at 16.3 min), free antibody (at 33 min), and the stabilizers (at 42 min), respectively. The pertuzumab conjugates 2B and 3B showed similar elution profile, except that the intensity of the free antibody peak was marginally higher and the free stabilizer peak observed at 42 min was found missing in electrostatic attachment (2B).

Physicochemical Characterization

Pertuzumab and trastuzumab AuNP conjugates, each prepared by both electrostatic and covalent procedures, were purified by centrifugation and separated by SEC column, and the physicochemical properties such as UV-Vis, TEM and hydrodynamic size measurements were compared.

UV-Vis:

The nanoconjugates prepared by both procedures showed surface plasmon resonance located at 525 nm. There was no change in UV-Vis peak position for conjugates prepared and isolated by the two procedures. There was no major change in location of the surface plasmon resonance peaks for the pegylated gold nanoparticles and gold antibody nanoconjugates.

TEM:

TEM image analyses of nanoconjugates obtained by electrostatic adsorption and covalent attachment showed mean core particle diameters of 16±8 nm and 16±4 nm, respectively. TEM images of electrostatically adsorbed and covalently bound antibody conjugated AuNPs purified by HPLC isolated by SEC procedure showed uniformly dispersed nanoparticles as compared to the conjugate isolated by centrifugation. TEM images of centrifuged conjugates displayed individual nanoparticles, indicating that these aggregates are remove by the centrifugation process. Comparing the TEM images, we hypothesize that the attachment of antibodies through physioadsorption cannot be eliminated, even if prepared through covalent attachment.

Hydrodynamic Diameter and Zeta Potential

The hydrodynamic diameter and zeta potential values obtained by dynamic light scattering measurements for citrate stabilized, pegylated AuNPs followed by antibody conjugation were compared. There was a ˜10 nm increase in hydrodynamic diameter of pegylated-AuNP conjugates over citrate coated AuNPs, and the zeta potential shifted to a more negative value, thereby confirming pegylation. The hydrodynamic diameter was found to be increased at each step.

An overlay of the hydrodynamic diameter profiles were also prepared for each human antibody-AuNP conjugate. The increased diameter observed for conjugates suggests that there is physioadsorbed antibody present in the covalently attached HPLC purified and SEC isolated conjugate. The zeta potential value of the conjugates synthesized by electrostatic attachment changed to a more negative value, while those synthesized by covalent attachment became neutral. However, the zeta potential values are dilution and solvent dependent; therefore, a conclusion cannot be drawn on the surface attachment based on these values alone.

Quantitative Protein Estimation

The protein estimation was carried out in these conjugates by performing a micro-Bradford assay. The extent of conjugation and the total amount of protein present in the conjugates prepared by electrostatic and covalent attachment were compared.

For conjugates isolated by centrifugation, supernatants obtained by three washings were used for protein assay. The amount of protein estimated by Bradford assay for the supernatants was subtracted from the total amount used for conjugation. Through Bradford assay, it was estimated that ˜40-50% of antibody was present in electrostatic pertuzumab and in covalently (3A) synthesized antibody gold conjugate pellets.

For conjugates purified by HPLC and isolated by SEC, the second fraction, associated with Peak 2 and corresponding to free antibody, was collected, then concentrated using a 50 KD filter and used for protein quantification.

The Bradford assay indicated presence of ˜60-70% of antibody in covalently attached conjugate (3B); whereas, for electrostatic conjugation (2B) the amount of protein present was similar to those isolated by the centrifugation process.

Assays were performed to compare the presence of protein content in the conjugate and to correlate these data with proteomics data. Therefore, upon comparing the presence of protein content, it is evident that the covalently conjugated antibody conjugate, purified by HPLC and isolated by SEC procedure and by centrifugation (3A and 3B), respectively, have relatively higher antibody abundance in comparison to values obtained for other conjugates.

Proteomic Analysis

Detailed proteomic analyses have been performed on the isolated conjugates. The spectral counts for both light and heavy chains were compared along with the sequence coverage. The spectral counts and coverage data indicate the relative abundance of the protein bound to gold nanoconjugate.

However, the isolation technique had an influence on the spectral counts and coverage data. HPLC purified and SEC isolated covalent conjugate (3B) shows relatively higher spectral counts and sequence coverage values for covalently bound conjugate when compared to electrostatic conjugation (2A, 2B), while the centrifuge purified conjugate (3A) shows a slight variation between the covalent and electrostatic conjugation, suggesting that HPLC purification aids in the removal of physioadsorbed antibody.

The spectral count and sequence coverage is similar for both covalently attached and electrostatically attached conjugate, indicating that the physioadsorption of antibody is inevitable and that the centrifuge procedure does not help in complete removal of free antibody.

Binding Affinity Studies

Enzyme-linked immunosorbent assay (ELISA) studies have been performed both by immuno-sandwich and direct methods with proper controls to validate the binding affinity of the gold antibody nanoconjugate. ELISA confirms relatively good binding for the covalently attached gold antibody conjugate purified by HPLC and isolated by SEC procedure compared to the conjugate obtained by centrifuged pellets. The electrostatic attachment showed minimal-to-no binding.

TEM Immuno-Histochemistry

Detailed immuno-histochemistry was performed to quantitate and validate the binding affinity of pertuzumab and trastuzumab conjugated to gold nanoparticles. In TEM imaging, anti-human IgG (6 nm) gold nanoconjugate was incubated with antibody gold nanoconjugate that was purified by HPLC and isolated by SEC procedure (3B), and compared with pegylated gold conjugate. The smaller size gold-anti-Human-IgG-conjugate 5 nm appear as light gray spots surrounding the dark 15 nm AuNP-PEG-PER conjugate indicating the affinity of the anti-human IgG antibodies towards the humanized antibody, pertuzumab surface conjugated to gold nanoparticle. The distances from the surface of AuNPs to the antibodies conjugated through the PEG linker were measured, and were found to be in the range about 16-18 nm.

Binding Affinity Studies

Dark field imaging was performed on fixed cells treated with gold antibody nanoconjugate to demonstrate the binding affinity of AuNP-PEG-PER on HER2 over-expressed SKBR-3 cells. The binding affinity of antibody nanoconjugates (both electrostatic (2A, 2B) and covalent attachment (3A, 3B)) purified by HPLC and isolated by SEC and centrifugation procedure have been compared.

The covalently attached gold antibody nanoconjugate shows higher uptake compared to the conjugates in which the antibody is electrostatically attached. This result suggests the gold antibody nanoconjugate has the ability to internalize in to the cells through effective HER2 binding.

The physioadsorbed conjugate showed minimal or no internalization suggesting that the conjugate is less stable in media.

Blocking studies using HER-2 followed by incubation with gold antibody nanoconjugate are still pending.

Cellular Uptake Studies

Detailed cellular uptake studies have been performed to understand the nature of interaction and mechanism of internalization of antibody gold nanoconjugate. SKBR-3 cells over-express HER2 receptors and therefore used to study the mechanism of internalization of gold antibody nanoconjugate.

The cells were incubated with antibody gold nanoconjugates synthesized by electrostatic and covalent attachment and the internalization process was compared. The amount of gold internalized in the cells for both conjugates (0.4 μg) purified by HPLC and isolated by SEC procedure are similar as determined by ICP-analysis, while those isolated by centrifuge was minimal.

TEM images suggest that covalently attached conjugates internalize through receptor mediated endocytosis (RME). A clear pit formation (size <200 nm) is observed in the membrane for the covalently bound conjugate validating RME.

There exists, however, a significant difference between covalent and electrostatic attached particles in the accumulation of particles within the cells.

For electrostatic conjugation large amount of nanoparticles were found to be accumulated in lysosomes and late endosomes. The amount of particles accumulated in cytoplasm was minimal.

For covalently conjugated nanoconjugate, large amount of particles are observed as aggregates in cytoplasm, while individual particles are found to be localized sparsely in lysosomes and endosomes. However, detailed investigations must to be carried out to determine if the endocytosis is clathrin or caveolae mediated.

Detailed Synthesis of Covalent and Electrostatic Conjugated Pertuzumab to AuNP-PEG2 (Au:PEG 1:2)

Gold nanoparticles (AuNPs) were pegylated with end terminal carboxyl groups at different stoichiometric ratios of Au—NP-PEG-COOH 1:2 and 1:5. Au:PEG 1:2 stiochiometric nanoparticles will be hence forward be referred to as AuNP-PEG2, and Au:PEG 1:5 as AuNP-PEGS. PEG is polyethylene glycol.

Activation of Carboxyl groups (1:2 ratio): Carboxyl groups in the AuNP-PEG2 were activated using 1-Ethyl-3-[3-dimethylaminopropyl] carbodiimide hydrochloride/N-hydroxysulfosuccinimide (EDC/NHS) chemistry using 4-morpholinoethanesulfonic acid (MES) buffer at pH 4.5. The EDC 10 mg and sulfo-NHS 10 mg were dissolved in 40 μl MES buffer, and then mixed together. This solution was added to the 1 ml of AuNP-PEG2 and incubated at 37° C. for 3 hrs with continuous shaking at 650 rpm. The activated AuNPs were centrifuged at 1000 revolutions per minute (rpm) for 10 min at room temperature (RT, i.e., 37° C. and excess of EDC/NHS was separated from the activated nanoparticles.

Antibody Conjugation: Pertuzumab 20 μg was added to the 200 μl of 1×PBS at pH 7.4. To this solution, the activated AuNP-PEG2 was added slowly in a drop-wise fashion. This reaction mixture was allowed to incubate at room temperature overnight with continuous shaking at 650 rpm. All reactions were performed in triplicates.

Purification by HPLC and isolation by size exclusion chromatography (SEC): After overnight incubation, the reaction mixture was purified by subjecting a 200 μl sample to size exclusion chromatography. The peaks were collected as PEAK 1: AuNP-Pertuzumab at 16 min (1.5 ml peak collected volume); PEAK 2: Free pertuzumab at 30 min (1.5 ml peak collected volume); PEAK 3: EDC/NHS, histidine and other antibody additives after 40 mins (2 ml peak collected volume).

Purification by Centrifugation: The reaction mixtures were centrifuged at 15,000 rpm for 20 min at 4° C. The supernatant was collected as Sup #1. The pellet was further washed two times by 1×PBS and Sup #2 and Sup #3 were collected.

Protein quantification using Bradford Assay: SEC peak 2 was collected as 1.5 ml. This 1.5 ml solution was concentrated to 200 μl by 50 kD filters and used for protein analysis. 1.5 ml was also used directly for protein analysis.

Results: All supernatants were also analyzed for detecting the amount of protein unconjugated to the nanoparticles. A standard curve was developed using serial dilutions of pertuzumab and unknown samples were analyzed. Based on the results obtained, it was found that approximately 60-70% of protein was conjugated to the AuNPs by covalent conjugation.

Electrostatic conjugation: The AuNP-PEG2 was incubated with pertuzumab 20 μg without using EDC/NHS. Rest of the procedure used for conjugation was exactly same as shown above. The reaction mixture was further purified by HPLC and isolated by SEC and by centrifugation, and protein quantification was performed by Bradford assay.

Results: In case of physioadsorption, approximately 50-60% protein was conjugated on the surface of AuNPs.

Trastuzumab—AuNP Conjugate Synthesis

Trastuzumab—AuNP conjugate synthesis was carried out using the same steps as provided for pertuzumab—AuNP conjugate synthesis, as described above. Results and outcomes for each step were similar.

Synthesis of Single Human Antibody Pertuzumab Bound to AuNP

Preparation of covalent bonded human antibody pertuzumab—AuNP conjugate, comprising the steps:

Preparation of citrate coated AuNPs in aqueous solution,

Preparation of AuNP-PEG-COOH in solution,

Activation of carboxyl terminated AuNP-PEG-COOH using EDC/Sulfo-NHS,

Addition of pertuzumab human antibody to solution containing AuNP-PEG with activated terminal groups solution,

Purification by HPLC, and Isolation of AuNP-PEG-PER Solution by SEC

Preparation of covalent bonded human antibody trastuzumab—AuNP conjugate, comprising the steps:

Preparation of citrate coated AuNPs in aqueous solution,

Preparation of AuNP-PEG-COOH in solution,

Activation of carboxyl terminated AuNP-PEG-COOH using EDC/Sulfo-NHS,

Addition of trastuzumab human antibody to solution containing AuNP-PEG with activated terminal groups solution,

Purification by HPLC, and isolation of AuNP-PEG-TRAS solution by SEC.

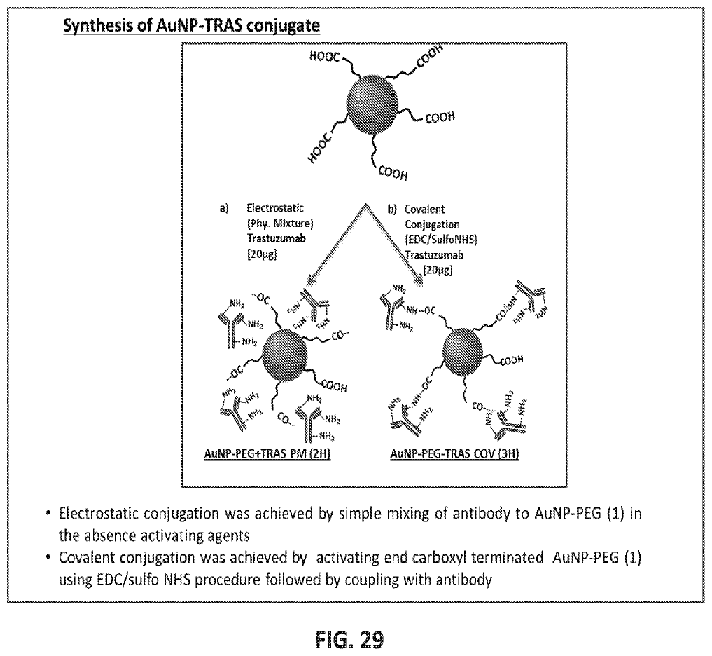

Results in Figures. The tests included both binding of pertuzumab and binding of trastuzumab to form nanoconjugates with gold. FIGS. 1-28 include data related to the pertuzumab nanoconjugate and FIGS. 29-39 include data related to the trastuzumab nanoconjugate.

FIG. 3 includes a comparison of the HPLC chromatograms of experimental nanoconjugates synthesized by both electrostatic and covalent attachment, including an overlay of AuNP-PER conjugate chromatograms prepared by both electrostatic (2B) and covalent (3B) procedures, along with a chromatogram for free monoclonal antibody used for conjugation.

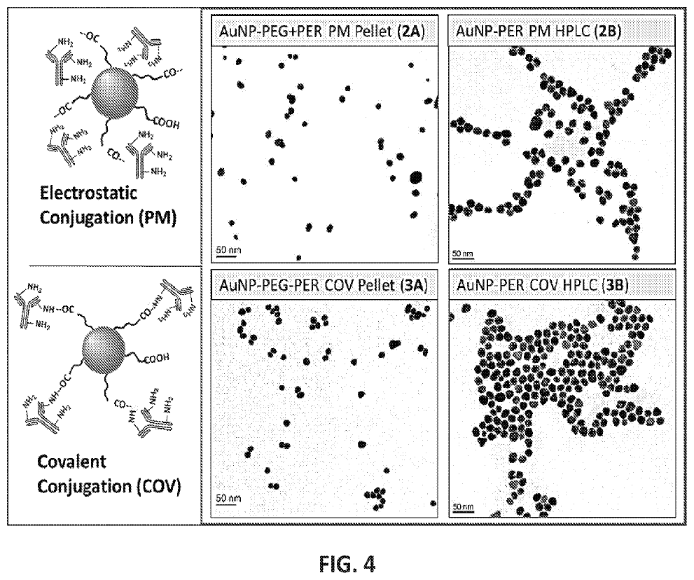

FIG. 4 shows images and schematic representations of experimental nanoconjugates 2A, 2B, 3A and 3B obtained by electrostatic adsorption and covalent attachment.

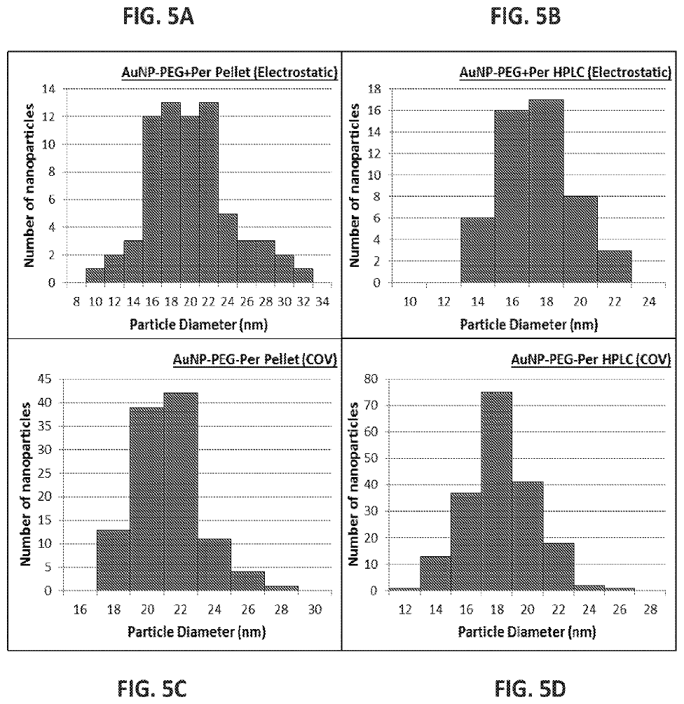

FIGS. 5A-5D include istograms respectively showing size distributions obtained from TEM for 2A formed by electrostatic conjugation, for 2B formed by electrostatic conjugation, for 3A formed by covalent conjugation, and for 3B formed by covalent conjugation.

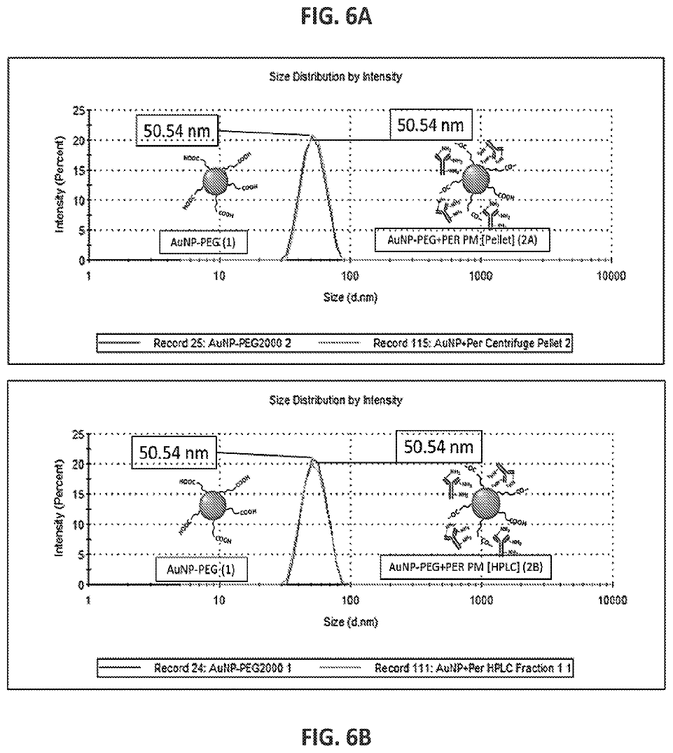

FIG. 6A includes super-imposed hydrodynamic diameter profiles for citrate stabilized gold pegylated AuNPs and for pegylated AuNPs with antibody conjugation formed by electrostatic conjugation (PM) with purification by conventional centrifugation (denoted by 2A). Pegylation was accompanied by an approximate 10 nm increase in hydrodynamic diameter when compared with non-pegylated AuNPs.

FIG. 6B includes Super-imposed hydrodynamic diameter profiles for citrate stabilized gold pegylated AuNPs and for pegylated AuNPs with antibody conjugation formed by electrostatic conjugation (PM) with purification by HPLC and isolation by SEC (denoted by 2B). Pegylation was accompanied by an approximate 10 nm increase in hydrodynamic diameter when compared with non-pegylated AuNPs.

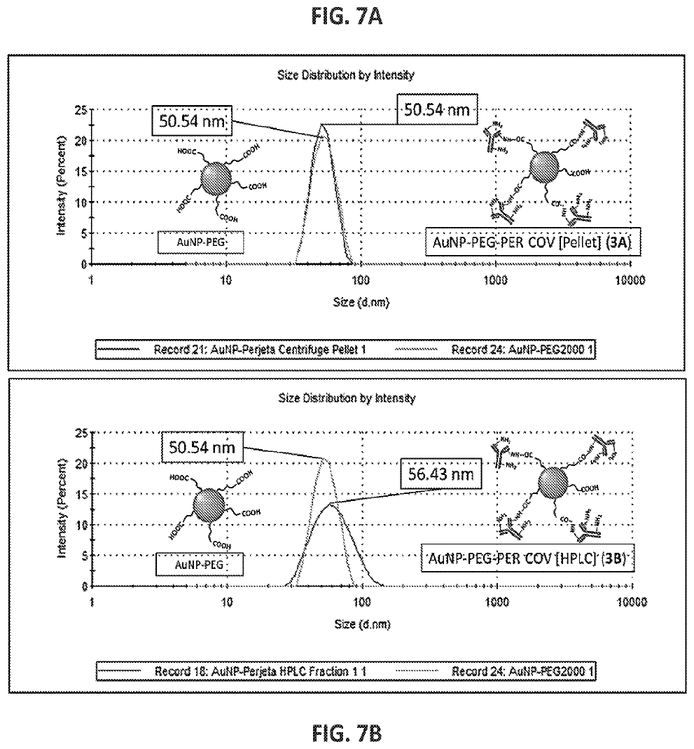

FIG. 7A includes Super-imposed hydrodynamic diameter profiles for citrate stabilized gold pegylated AuNPs and for pegylated AuNPs with antibody conjugation formed by covalent conjugation (COV) with purification by conventional centrifugation (denoted by 3A). Pegylation was accompanied by an approximate 10 nm increase in hydrodynamic diameter when compared with non-pegylated AuNPs.

FIG. 7B includes Super-imposed hydrodynamic diameter profiles for citrate stabilized gold pegylated AuNPs and for pegylated AuNPs with antibody conjugation formed by covalent conjugation (COV) with purification by HPLC and isolation by SEC (denoted by 3B).

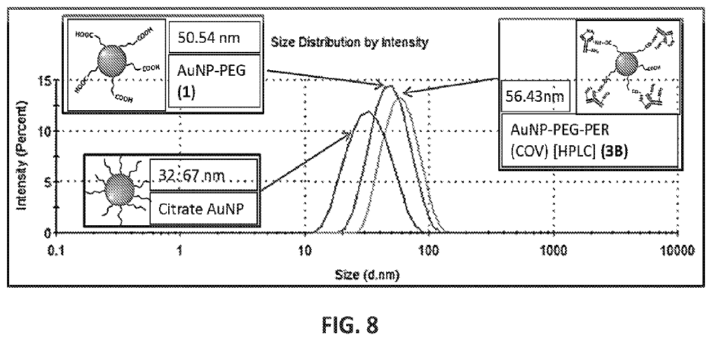

FIG. 8 includes super-imposed hydrodynamic diameter profiles for citrate stabilized (non-pegylated) AuNPs, for citrate stabilized pegylated AuNPs, and for pegylated AuNPs with antibody conjugation formed by covalent conjugation (COV) with purification by HPLC and isolation by SEC (denoted by 3B). Pegylation was accompanied by an approximate 10 nm increase in hydrodynamic diameter when compared with non-pegylated AuNPs. The increased diameter of approximately 24 nm (over non-pegylated AuNPs) for nanoconjugate 3B suggests presence of a physioadsorbed antibody in the covalently attached HPLC purified and SEC isolated nanoconjugate.

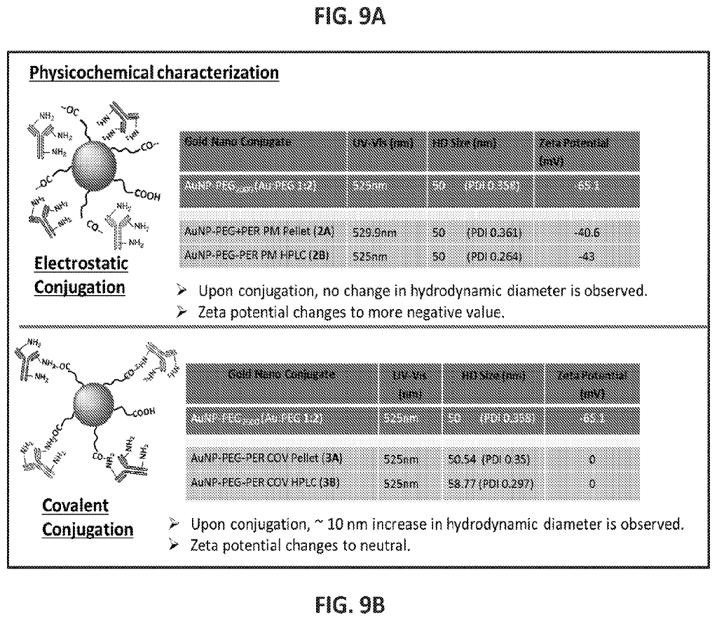

FIG. 9A includes measured values and summary statements of several observed changes for six nanoconjugates that were formed and purified by different procedures, as identified.

FIG. 9B includes Measured values that include locations of plasmon adsorption peak in UV-Vis spectra, hydrodynamic diameters, and zeta potentials for each nanoconjugate.

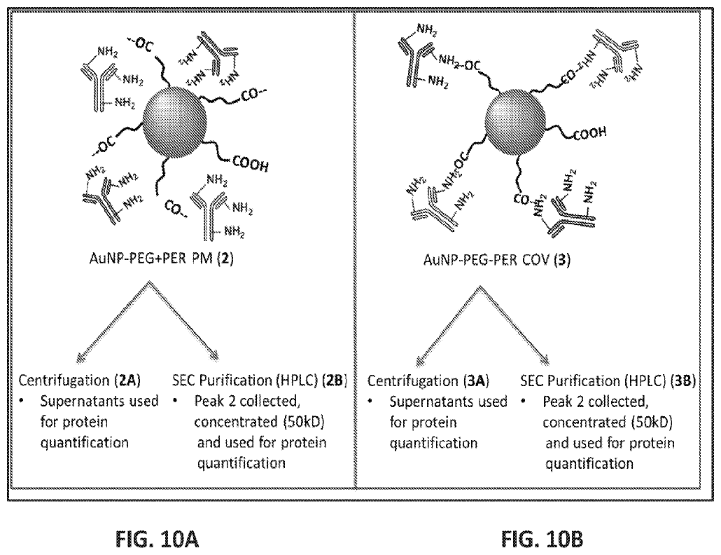

FIGS. 10A-10B are schematic diagrams illustrating the nanoconjugate for which protein quantification was measured, including electrostatic bonded constructs purified by two different means; namely, centrifugation and purification by HPLC and isolation by SEC, and covalent bonded constructs also purified by two different means; namely, by centrifugation and by HPLC purification and SEC isolation.

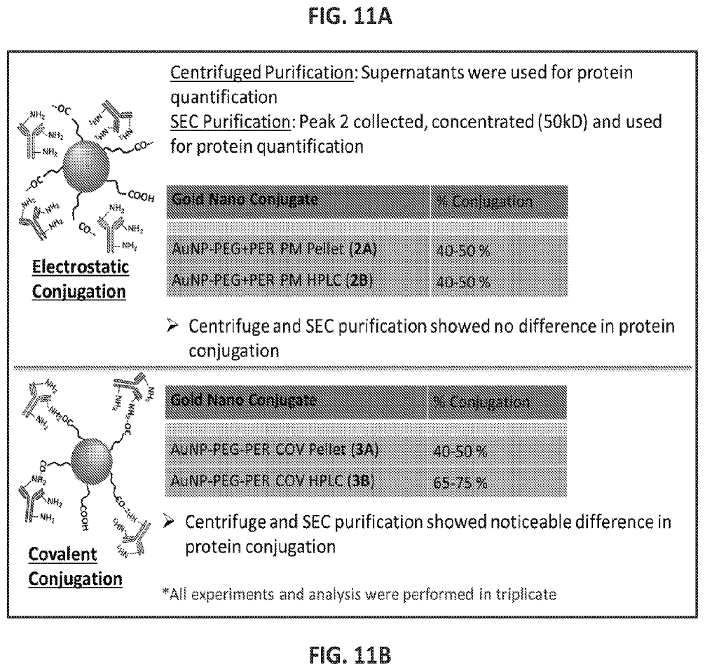

FIGS. 11-11B are easured values for protein present in the four identified nanoconjugates, prepared by either electrostatic or covalent methods and purified by either centrifugal or HPLC methods.

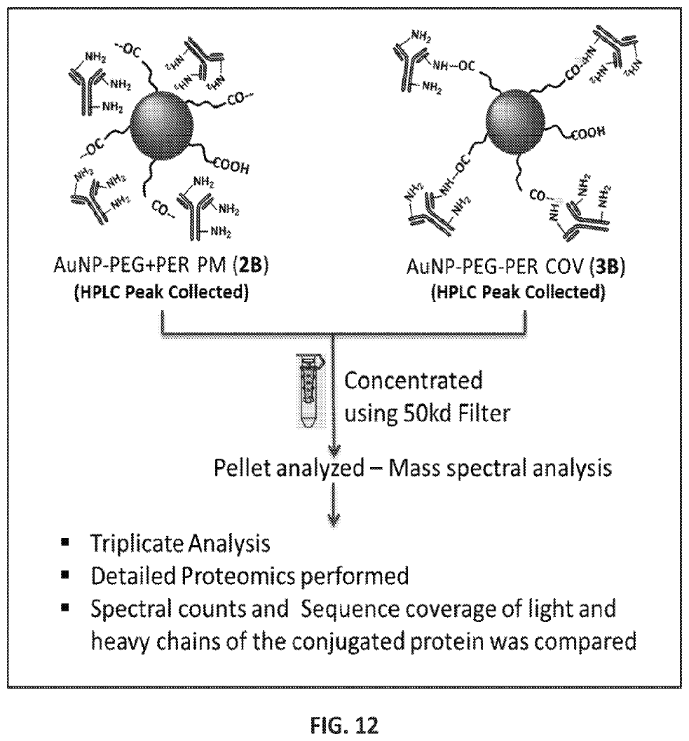

FIG. 12 is a schematic diagram of a preferred method for proteomic analyses of nanoconjugates formed by the electrostatic method and by the covalent method.

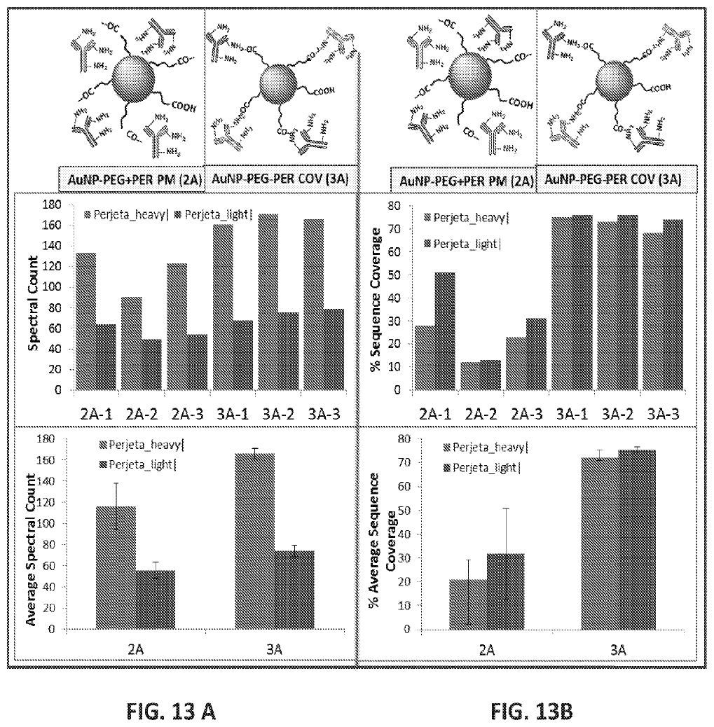

FIGS. 13A-13B are istograms showing spectral count, average spectral count, % sequence coverage, and % average sequence coverage for heavy and light chains for several gold nanoparticle—pertuzumab nanoconjugates, including those formed by the electrostatic method and by the covalent method using centrifuge isolation.

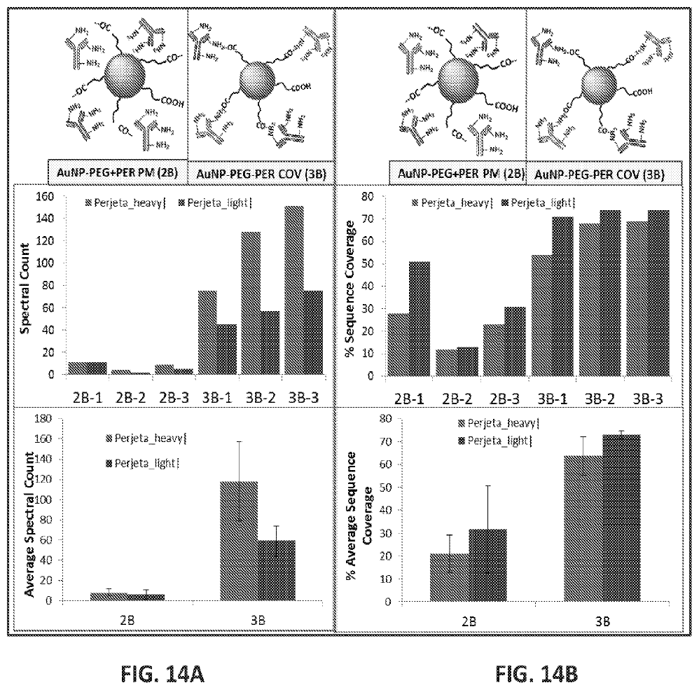

FIGS. 14A-14B are istograms showing spectral count, average spectral count, % sequence coverage, and % average sequence coverage for heavy and light chains for several gold nanoparticle—pertuzumab nanoconjugates, including those formed by the electrostatic method and by the covalent method using HPLC isolation.

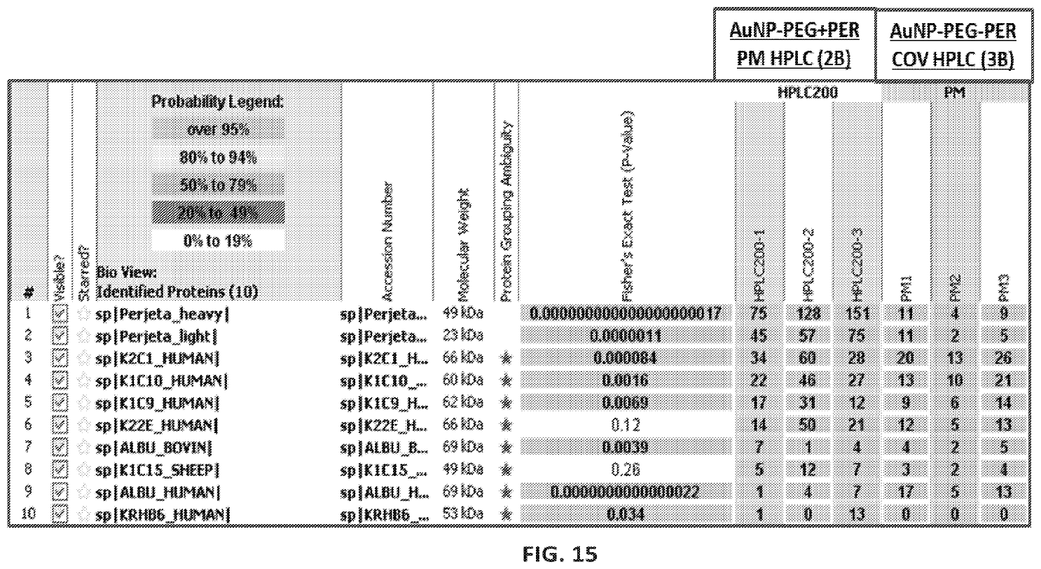

FIG. 15 are sequence coverage results from proteomics analysis, for both heavy and light chains of pertuzumab conjugated to gold nanoparticles.

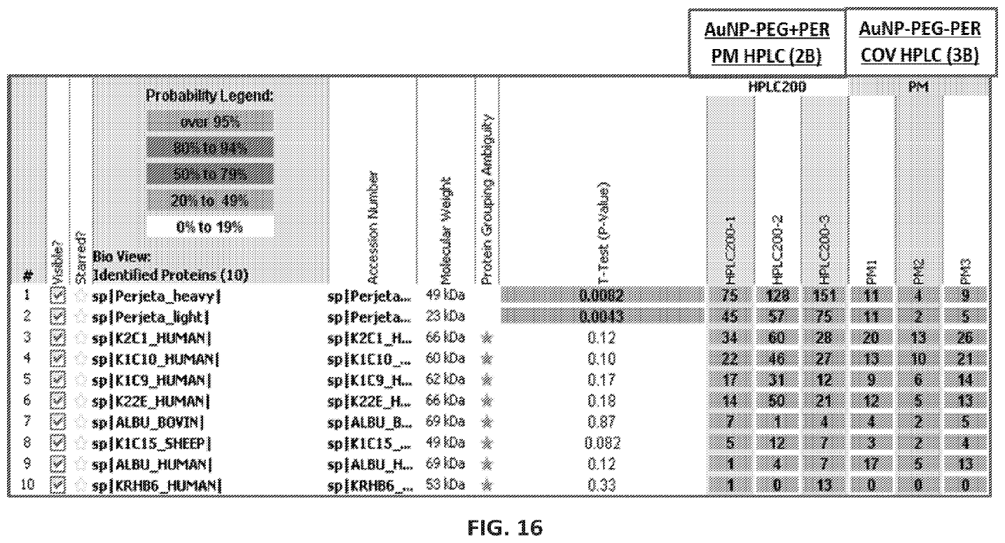

FIG. 16 are sequence coverage results from proteomics analysis, for both heavy and light chains of pertuzumab conjugated to gold nanoparticles.

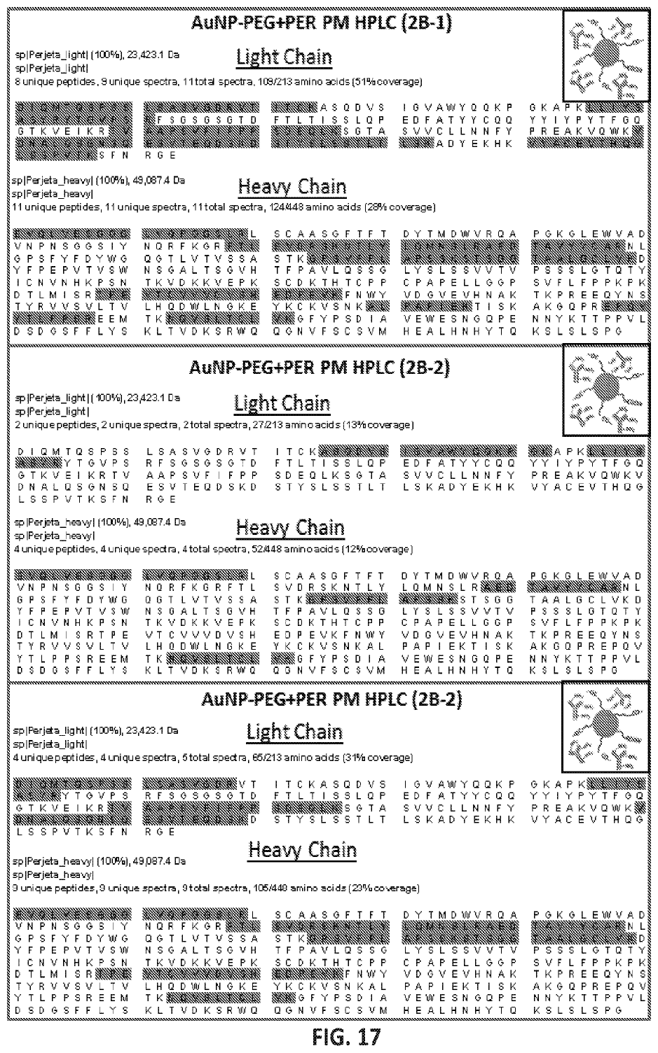

FIG. 17 are sequence coverage results from proteomics analysis, for both heavy and light chains of pertuzumab conjugated to gold nanoparticles.

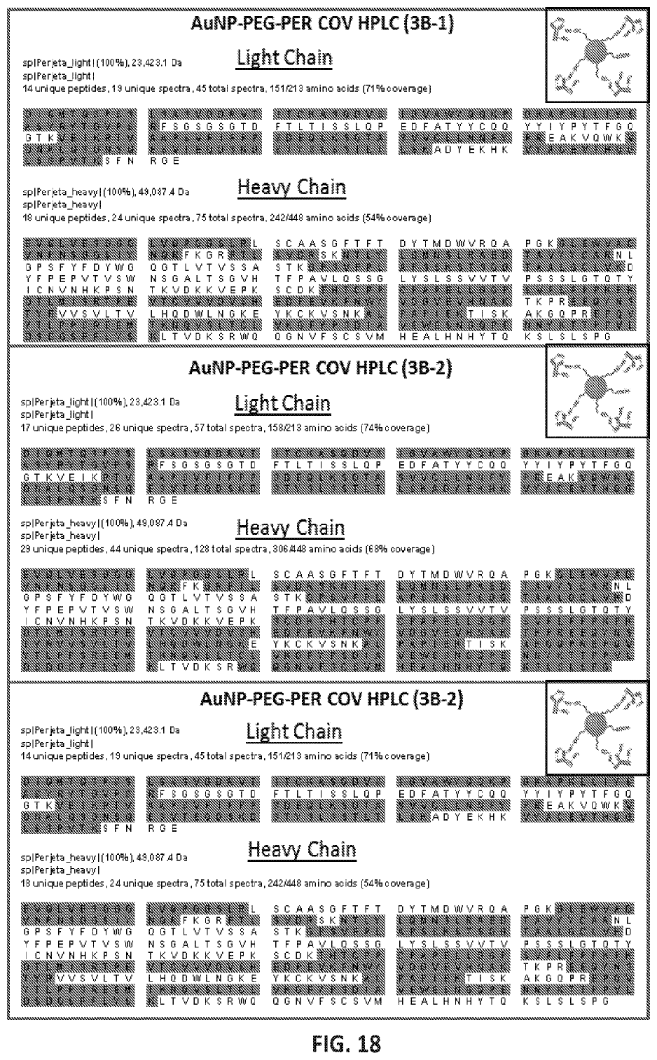

FIG. 18 are sequence coverage results from proteomics analysis, for both heavy and light chains of pertuzumab conjugated to gold nanoparticles.

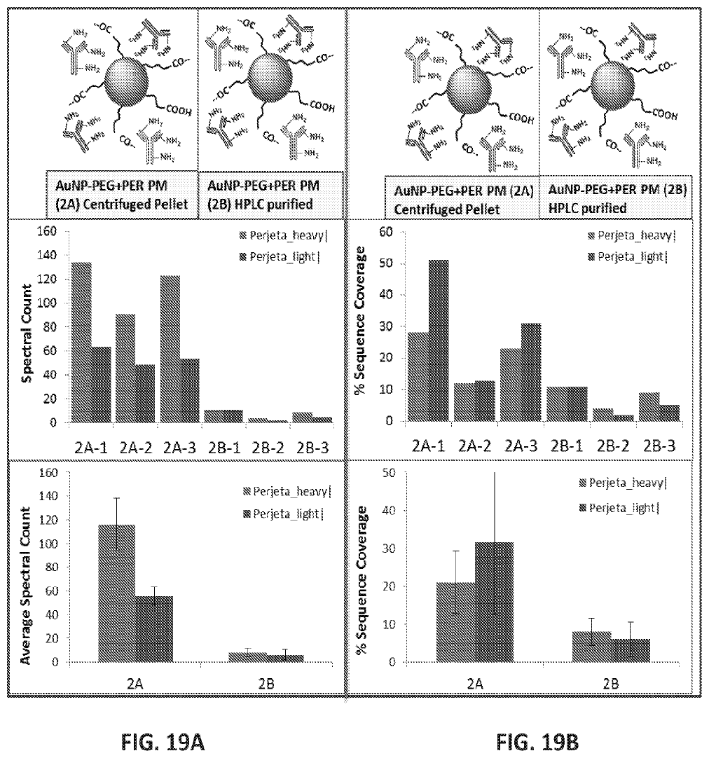

FIGS. 19A-19B are a comparison of spectral count, average spectral count, % sequence coverage, and % average sequence coverage for heavy and light chains, for centrifuged and for purified by HPLC and isolated by SEC nanoconjugates prepared by electrostatic (PM) conjugation method.

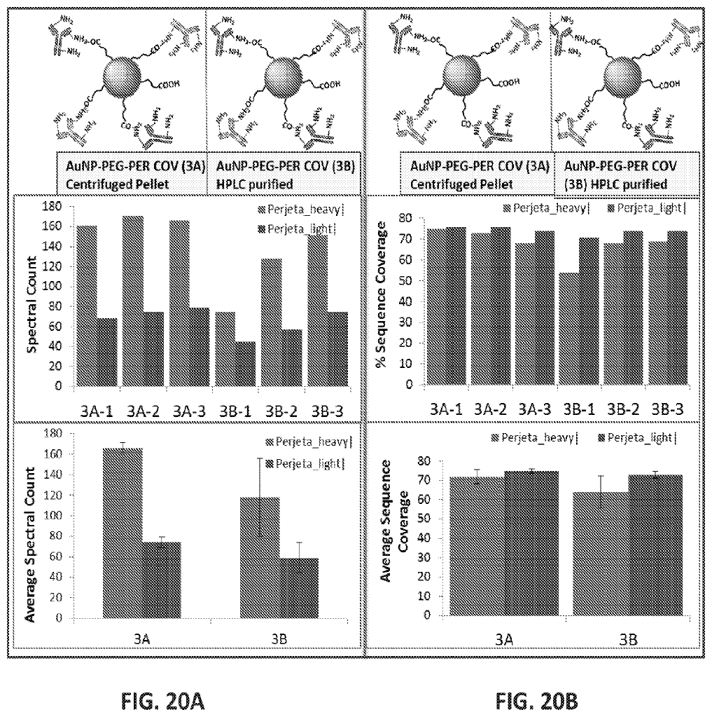

FIGS. 20A-20B are a comparison of spectral count, average spectral count, % sequence coverage, and % sequence coverage for heavy and light chains, for centrifuged and for HPLC purified and SEC isolated nanoconjugates prepared by covalent conjugation method.

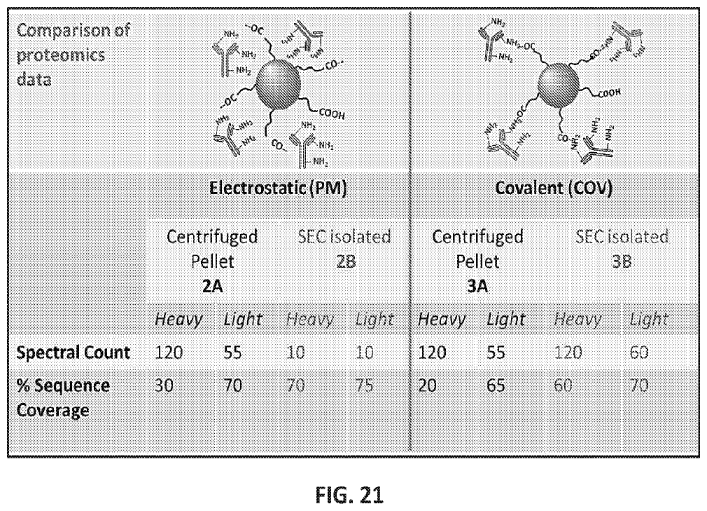

FIG. 21 is a comparison of spectral counts and sequence coverage obtained from proteomics data for nanoconjugates prepared by electrostatic (PM) and covalent conjugation methods.

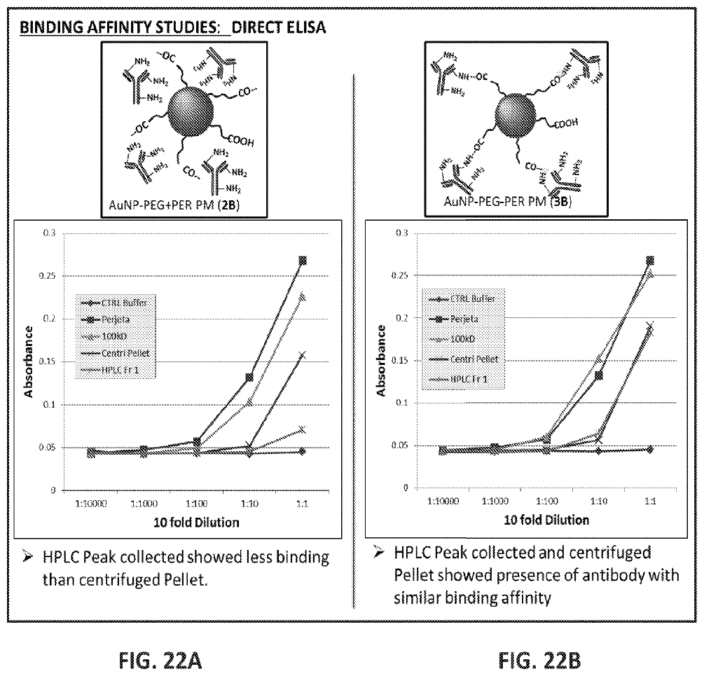

FIGS. 22A-22 are data of binding affinity data for pertuzumab conjugated to gold nanoparticles, obtained by direct ELISA, for nanoconjugates prepared by electrostatic (PM) and covalent conjugation methods.

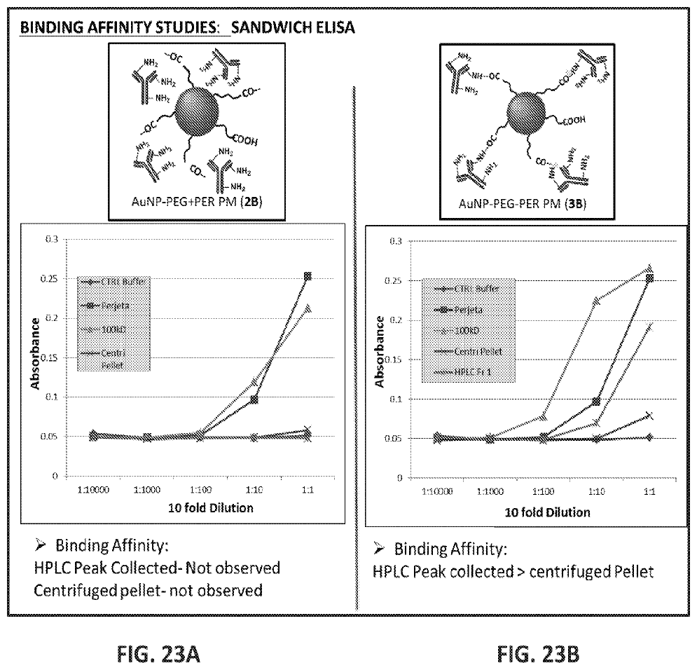

FIGS. 23A-23B are data of binding affinity data for pertuzumab conjugated to gold nanoparticles, obtained by sandwich ELISA, for nanoconjugates prepared by electrostatic (PM) and covalent conjugation methods.

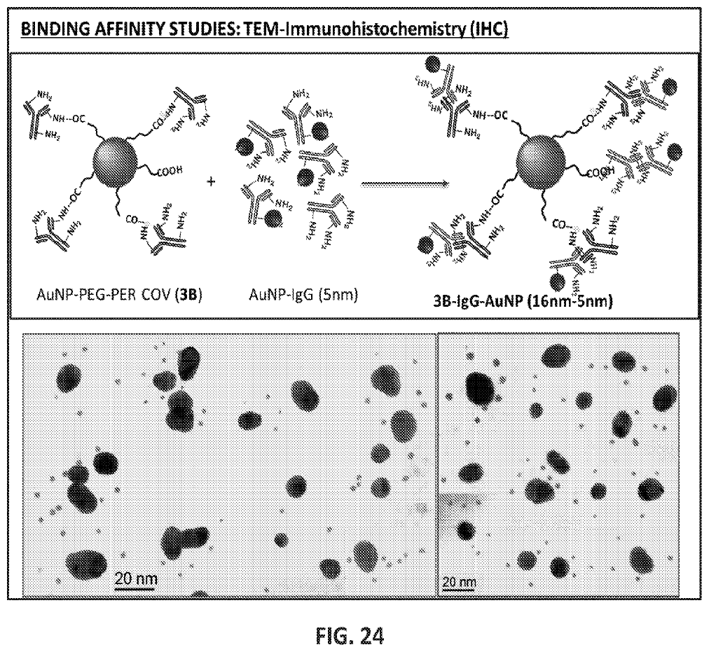

FIG. 24 includes related TEM images obtained from immuno histochemistry studies that validate the binding affinity of 3B antibody pertuzumab AuNP nanoconjugates purified by HPLC isolated by SEC procedure (large 15 nm dark spots) with anti-human IgG AuNP nanoconjugate (smaller 5 nm light grey spots). The upper panel illustrates attraction of these two entities. The final separation distance measured from an AuNP surface (through a PEG linker) to an anti-human AuNP IgG was approximately 16-18 nm.

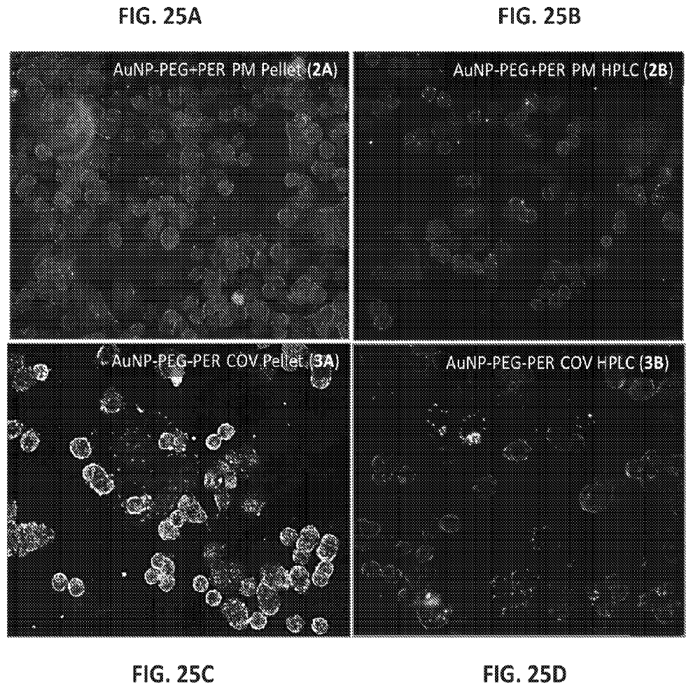

FIGS. 25A-25D are dark field microscope images for covalently attached AuNP pertuzumab nanoconjugates (FIGS. 25A-B) and electrostatically attached AuNP pertuzumab nanoconjugates (FIGS. 25C-D) that demonstrate their binding affinity to HER2 positive SkBr3 breast cancer cells. The covalently attached gold-antibody nanoconjugate showed higher uptake and greater internalization through effective HER2 binding than did the physioadsorbed gold-antibody nanoconjugate that was formed by electrostatic attachment, with the latter showing minimal or no internalization.

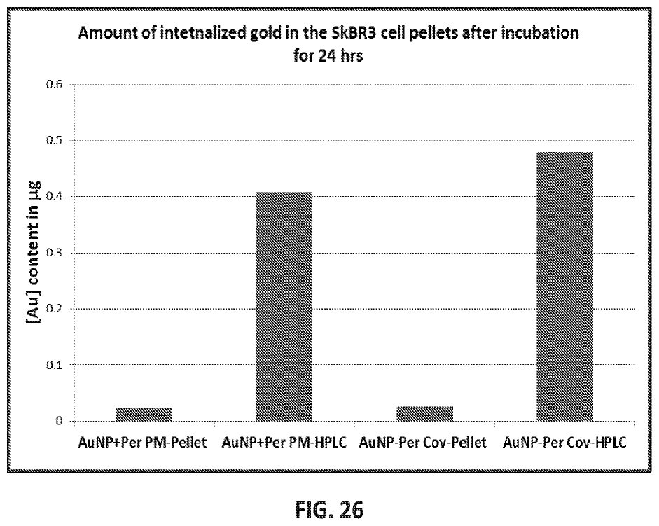

FIG. 26 is data concerning cellular uptake, as measured in Au content in μg using ICP analysis, for SKBR-3 cells with over expressed HER2 receptors when exposed to (pertuzumab) antibody—gold nanoparticle nanoconjugates for 24 hours, where the nanoconjugates were formed and isolated by four different methods: electrostatic attachment and centrifuge separation (PM-PELLET), electrostatic attachment and HPLC purification and SEC isolation (PM-HPLC), covalent attachment and centrifuge separation (COV-PELLET), covalent attachment and HPLC purification and SEC isolation (COV-HPLC).

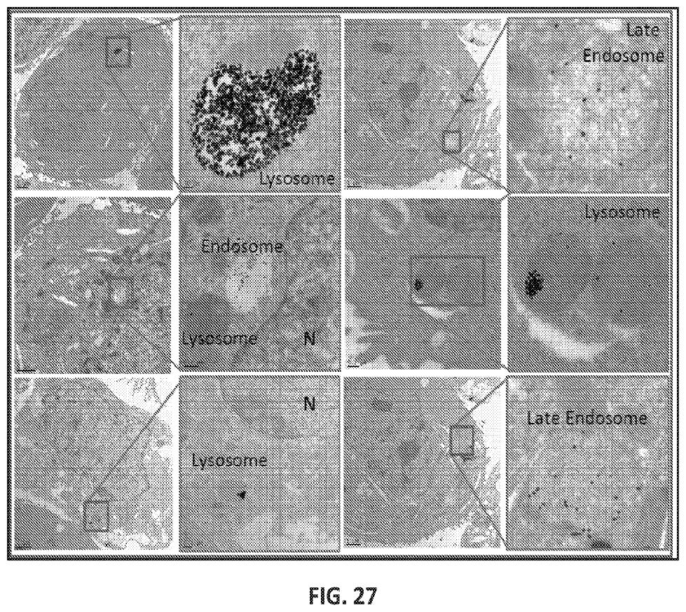

FIG. 27 includes TEM imaging studies illustrating cellular uptake for electrostatically formed nanoconjugates. A large fraction of electrostatically formed conjugated nanoconjugates were found to be located in lysosomes and late endosomes, whereas the amount located in cytoplasm was minimal.

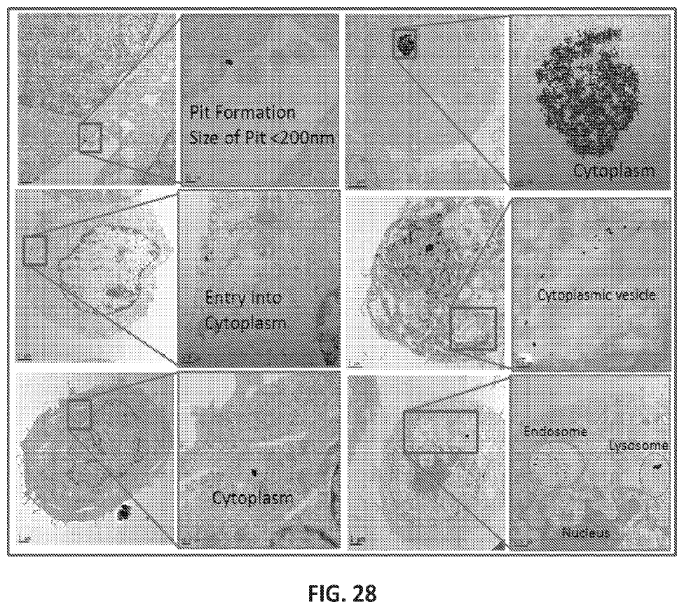

FIG. 28 includes TEM imaging studies illustrating cellular uptake for covalently formed nanoconjugates. A large fraction of covalently formed conjugated nanoconjugates were observed as aggregates located in cytoplasm.

FIG. 29 is a schematic showing steps in preferred syntheses of trastuzumab gold antibody nanoconjugates, similar to the FIG. 1 procedure. Syntheses were performed following two different procedures; namely, by electrostatic attachment (schematically represented in left branch), and by covalent conjugation (schematically represented in right branch).

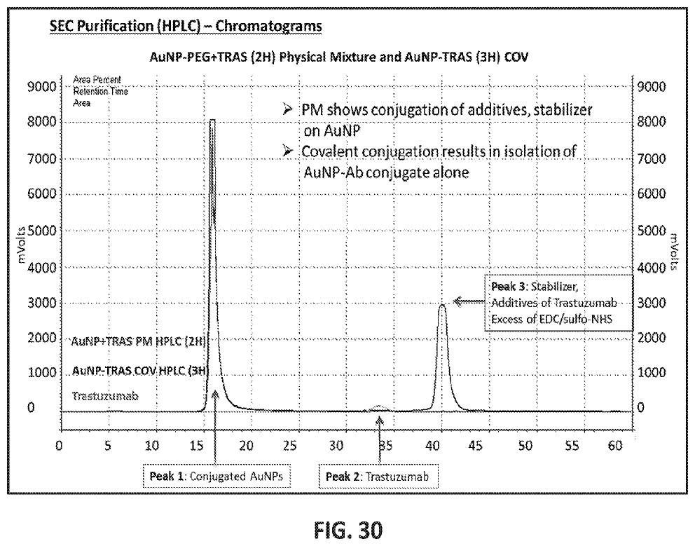

FIG. 30 is a comparison of the HPLC chromatograms of trastuzumab nanoconjugates synthesized by both electrostatic and covalent attachment. Shown is an overlay of AuNP-PER conjugate chromatograms prepared by both electrostatic (2B) and covalent (3B) procedures, along with a chromatogram for free monoclonal antibody used for conjugation.

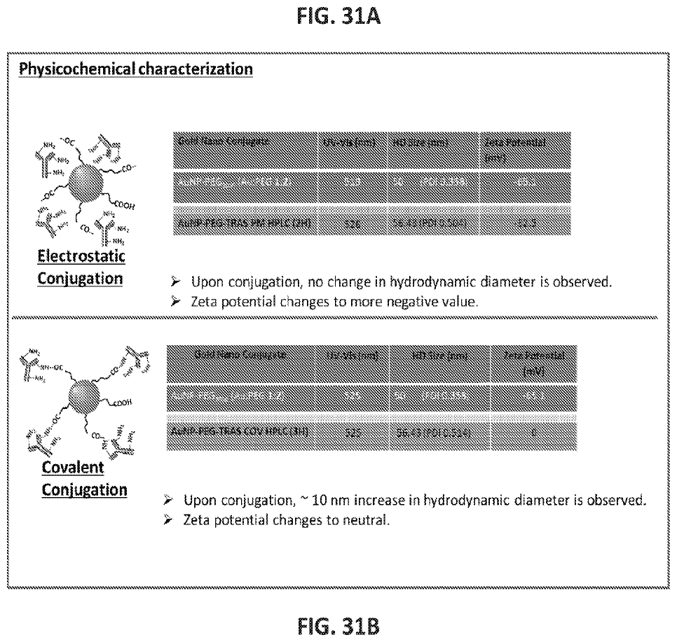

FIGS. 31A-31B include measured values and summary statements of several observed changes for six nanoconjugates that were formed and purified by different procedures, as identified. Measured values include locations of plasmon adsorption peak in UV-Vis spectra, hydrodynamic diameters, and zeta potentials for each trastuzumab nanoconjugate.

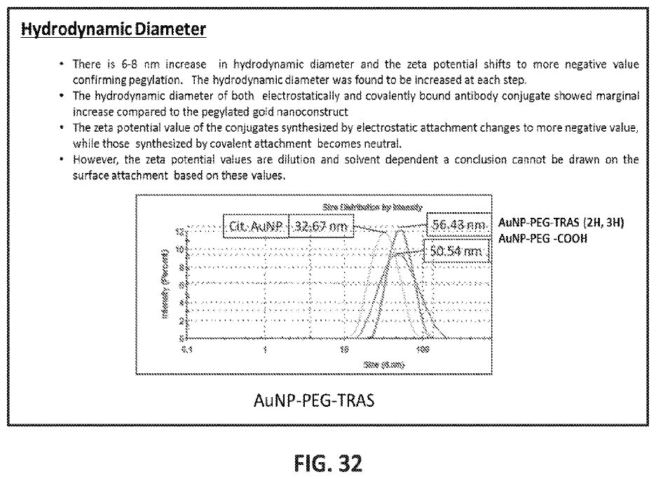

FIG. 32 includes Super-imposed hydrodynamic diameter profiles for citrate stabilized (non-pegylated) AuNPs, for citrate stabilized pegylated AuNPs, and for pegylated AuNPs with antibody conjugation formed by covalent conjugation (COV) with purification by HPLC and isolated by SEC (denoted by 3B). Pegylation was accompanied by an approximate 10 nm increase in hydrodynamic diameter when compared with non-pegylated AuNPs. The increased diameter of approximately 24 nm (over non-pegylated AuNPs) for nanoconjugate 3B suggests presence of a physioadsorbed antibody in the covalently attached purified by HPLC and isolated by SEC nanoconjugate.

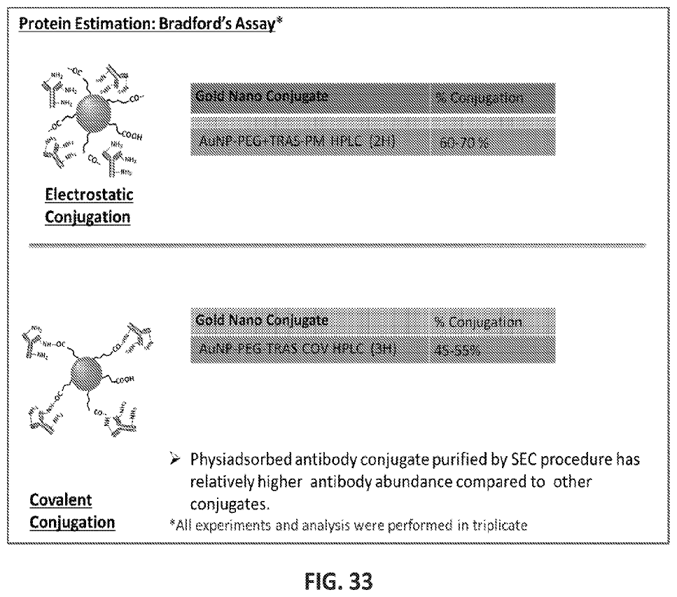

FIG. 33A-33B include measured values for protein present in the four identified trastuzumab conjugates, prepared by either electrostatic or covalent methods and purified by either centrifugal or HPLC purified and SES isolatedmethods, are listed.

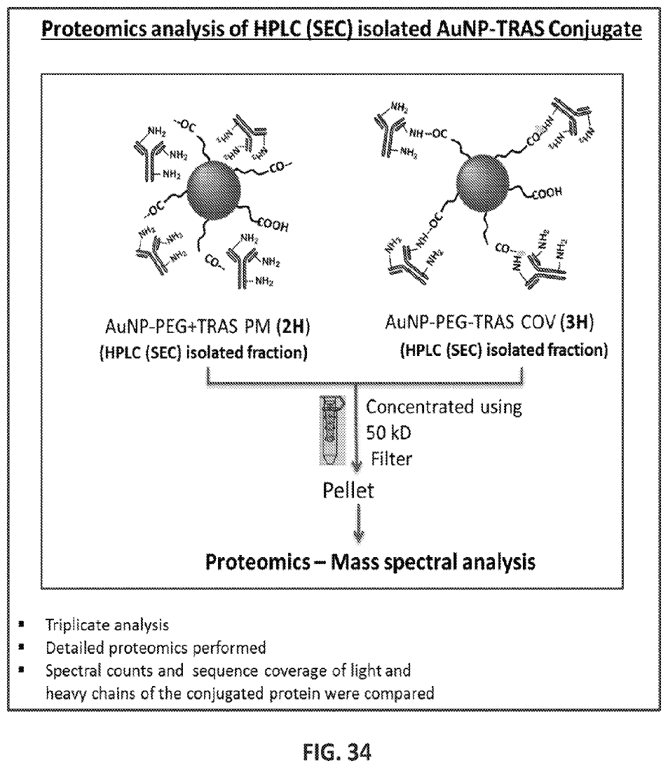

FIG. 34 includes schematic illustrating the process followed for proteomic analyses of trastuzumab nanoconjugates formed by the electrostatic method and by the covalent method.

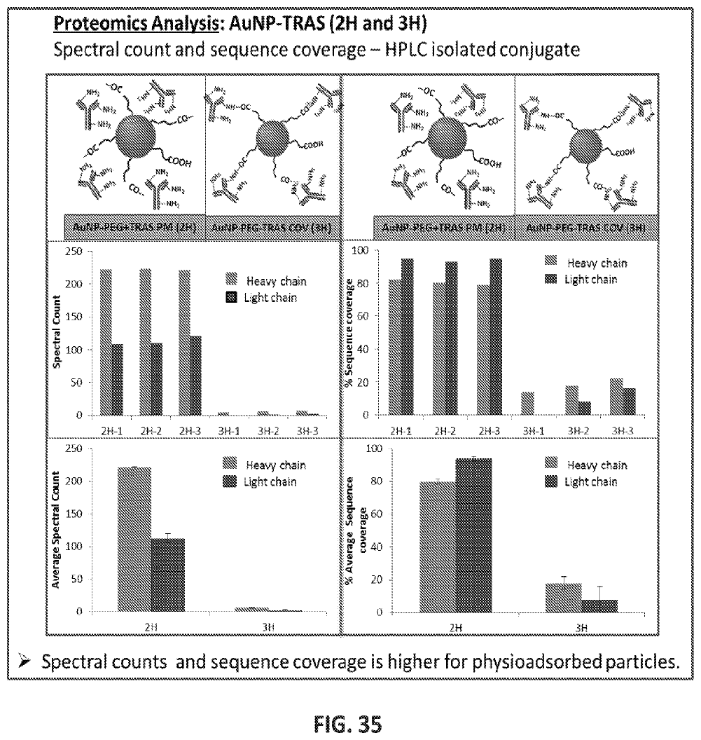

FIG. 35 includes histograms showing spectral count, average spectral count, % sequence coverage, and % average sequence coverage for heavy and light chains for several gold nanoparticle-trastuzumab nanoconjugates, including those formed by the electrostatic method and by the covalent method using centrifuge isolation.

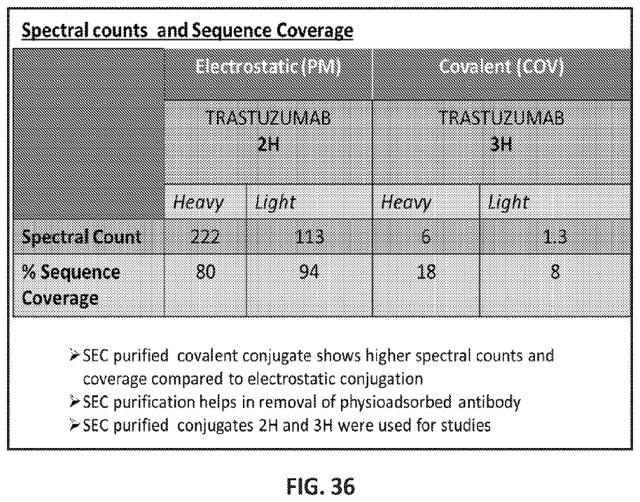

FIG. 36 includes spectral counts and sequence coverage for two methods of conjugation (electrostatic and covalent), and for each of the two conjugation methods are listed the values for spectral counts and sequence coverage for heavy chains and for light chains.

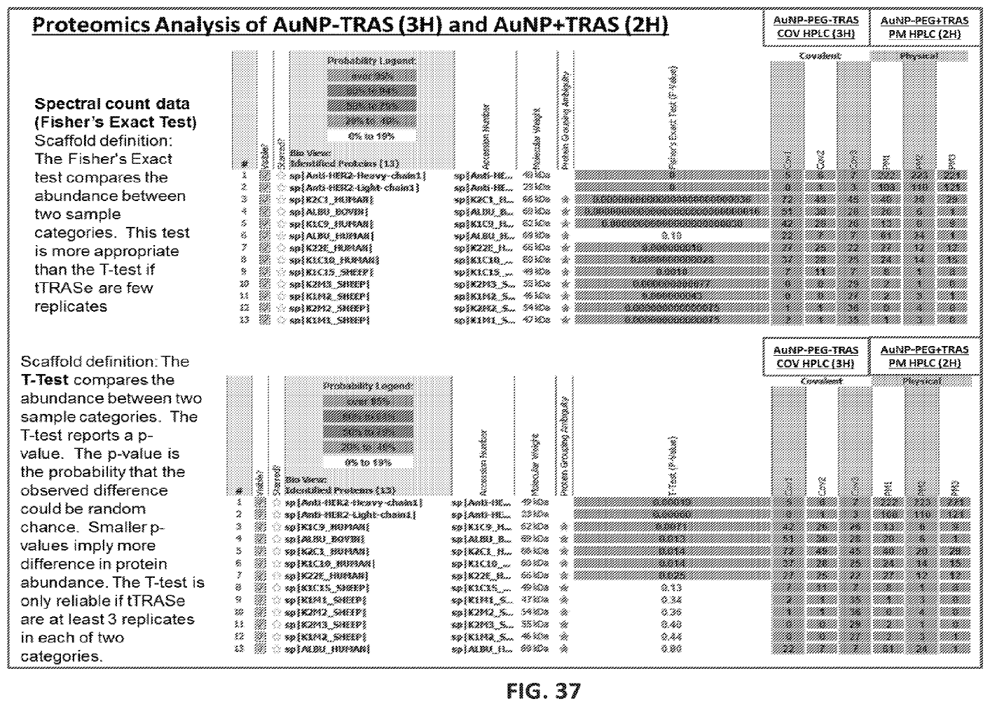

FIG. 37 includes sequence coverage results from proteomics analysis, for both heavy and light chains of trastuzumab gold nanoconjugates.

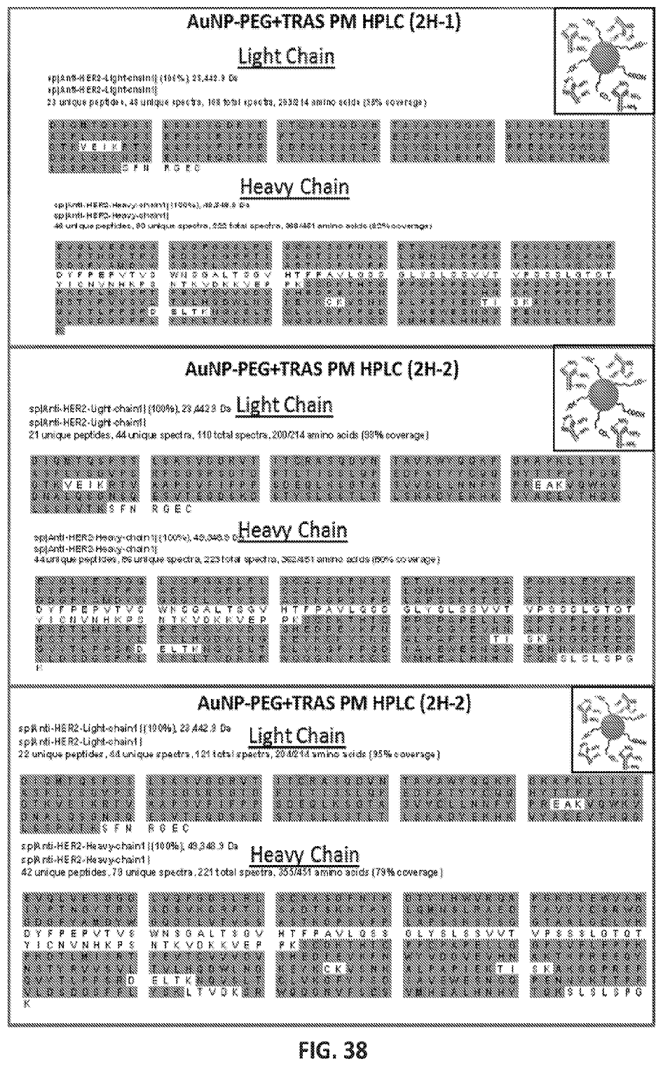

FIG. 38 includes sequence coverage results from proteomics analysis, for both heavy and light chains of trastuzumab gold nanoconjugates.

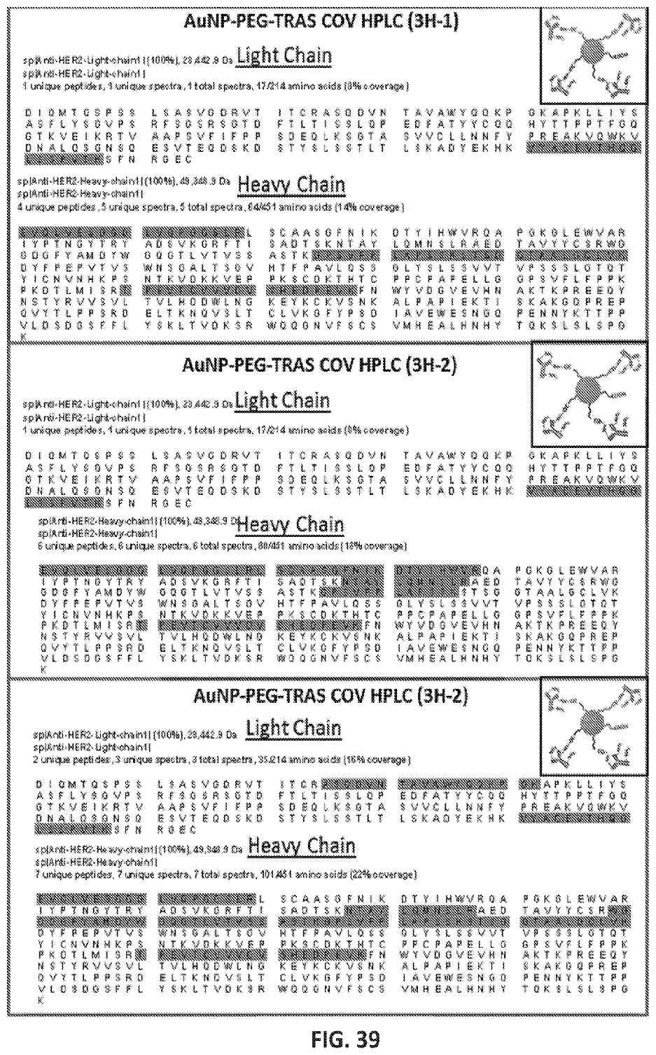

FIG. 39 includes sequence coverage results from proteomics analysis, for both heavy and light chains of trastuzumab gold nanoconjugates.

The physicochemical characterization permits efficacy deremintation. In a method of formation of nanoparticle conjugates for cancer diagnostics, imaging and therapy, the method allows determination a priori the efficacy of the nanoparticle conjugate for treatment of disease comprises following steps: Synthesis of nanoparticles followed by attachment of a linker and coupling of antibody of single type to nanoparticles to for covalent or electrostatic bonds. Then the physicochemical characterization is conducted. Either electrostatic or covalent bonding as may be chosen such that the original properties of the single antibody type are most fully retained for treatment of a particular disease.

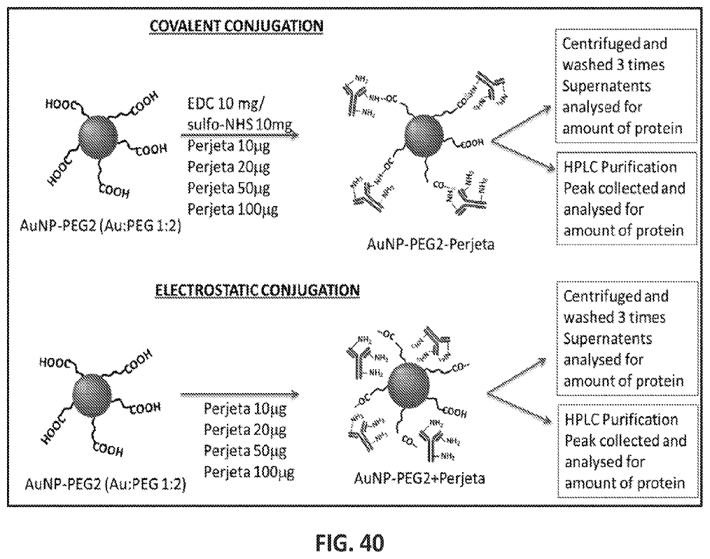

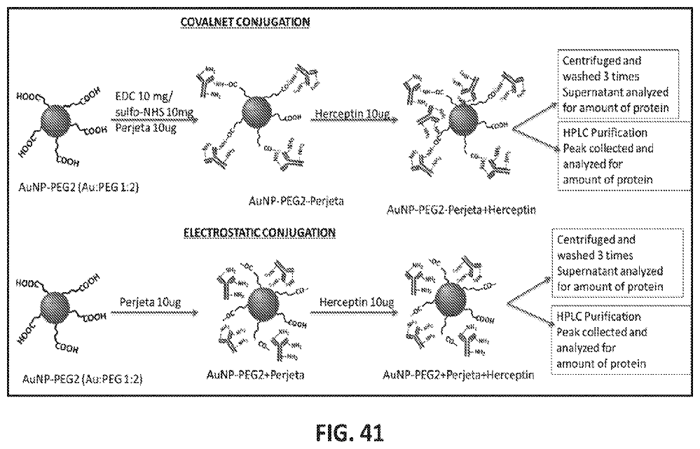

The optimization of the ratio of antibodies to nanoparticles is also part of preferred embodiments predetermine efficacy and form therapeutic agents. In order to optimize the best possible ratio of antibodies to the nanoparticles, experiments were conducted. Gold nanoparticles with a PEG linker of molecular weight 2000 with an end terminal carboxyl group were used at fixed concentration of 1:2 ratio of Au to the linker. These nanoparticles were used as a precursor to optimize the amount of antibody that can be sufficiently accommodated on the surface of nanoparticles. Varied amounts of antibody were used in the reaction such as 10 μg, 20 μg, 50 μg, and 100 μg. Both electrostatic and covalent conjugation was performed with single and with dual antibodies as shown in FIG. 40.

For electrostatic conjugation, the linker attached nanoparticles were incubated with 10 μg, 20 μg, 50 μg, and 100 μg of antibody and were subjected to purification techniques for subsequent analysis of protein conjugation using Bradford assay. In covalent conjugation, the end terminal carboxyl groups were activated using N-(3-Dimethylaminopropyl)-N′-ethylcarbodiimide hydrochloride (EDC) and N-Hydroxysulfosuccinimide sodium salt in 4-morpholinoethansulfonic acid buffer (pH4.5) followed by coupling with antibody 10 μg, 20 μg, 50 μg, and 100 μg. The antibody conjugated nanoparticles obtained were purified by both centrifugation and by size exclusion chromatography (SEC) techniques. The following table includes characterization of said nanoparticles.

TABLE 1
Physicochemical characterization and protein quantitation.

| | | | | |
|---|---|---|---|---|
| | | | | % of Protein Conjugation |
| | | | Zeta | by Bradford Assay and |
| Nanoconstruct | UV-Vis | Size (nm) | (mV) | Microdrop Analysis |

Covalent Conjugation

| | | | | | |
|---|---|---|---|---|---|
| AuNP − PEG (1:2) | 525 nm | 50 | (PDI 0.358) | −65.1 | — |
| AuNP − Per (10 μg) | 525 nm | 43.82 | (PDI 0.35) | −43 | 50-60 |
| AuNP − Per (20 μg) | 525 nm | 58.77 | (PDI 0.297) | −12 | 60-70 |
| AuNP − Per (50 μg) | 525 nm | 56.43 | (PDI 0.507) | −16 | 60-70 |
| AuNP − Per (100 μg) | 525 nm | 56.43 | (PDI 0.315) | −13 | 55-65 |

Electrostatic Conjugation

| | | | | | |
|---|---|---|---|---|---|
| AuNP + Per (10 μg) | 525 nm | 50 | (PDI 0.264) | −43 | 50-60 |
| AuNP + Per (20 μg) | 529.9 nm | 54 | (PDI 0.361) | −22.4 | 60-70 |
| AuNP + Per (50 μg) | 525 nm | 53.34 | (PDI 0.547) | −27.6 | 60-70 |
| AuNP + Per (100 μg) | 525 nm | 53.34 | (PDI 0.486) | −39.6 | 55-65 |

It was found that when 20 μg of antibody was used for protein conjugation, maximum conjugation efficiency was achieved and therefore, it was concluded that 20 μg of antibody would be preferred to achieve complete conjugation to AuNPs.

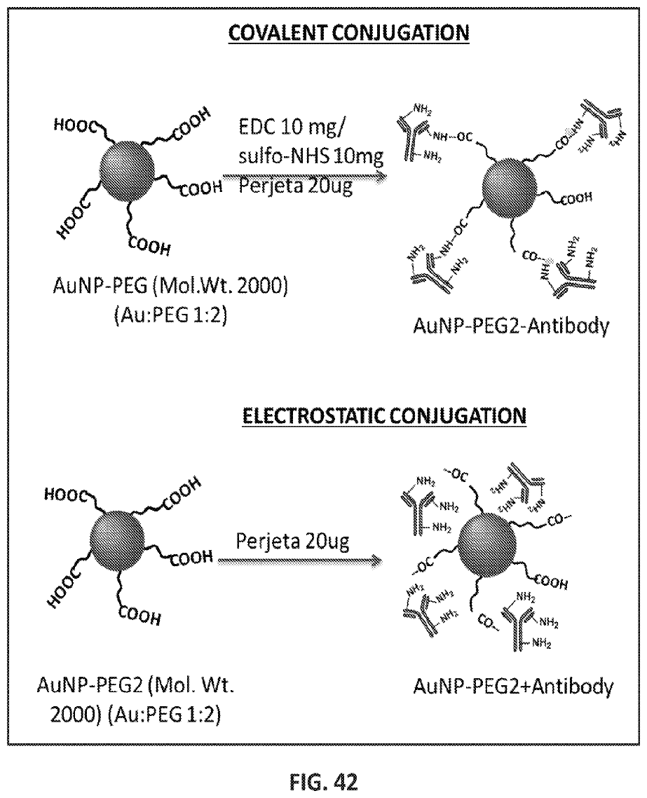

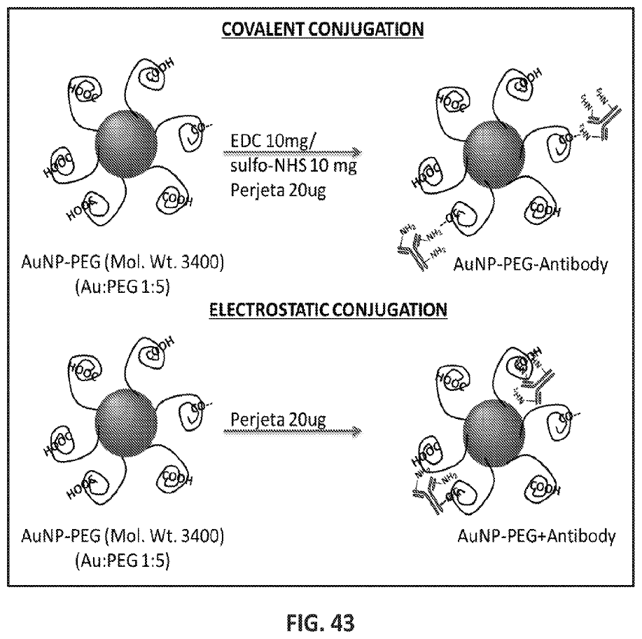

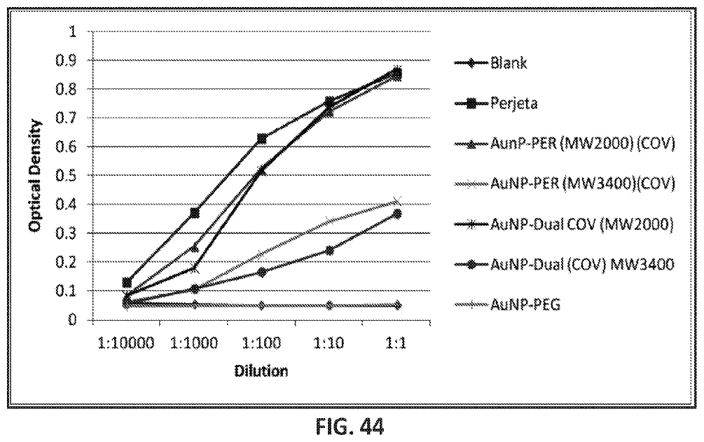

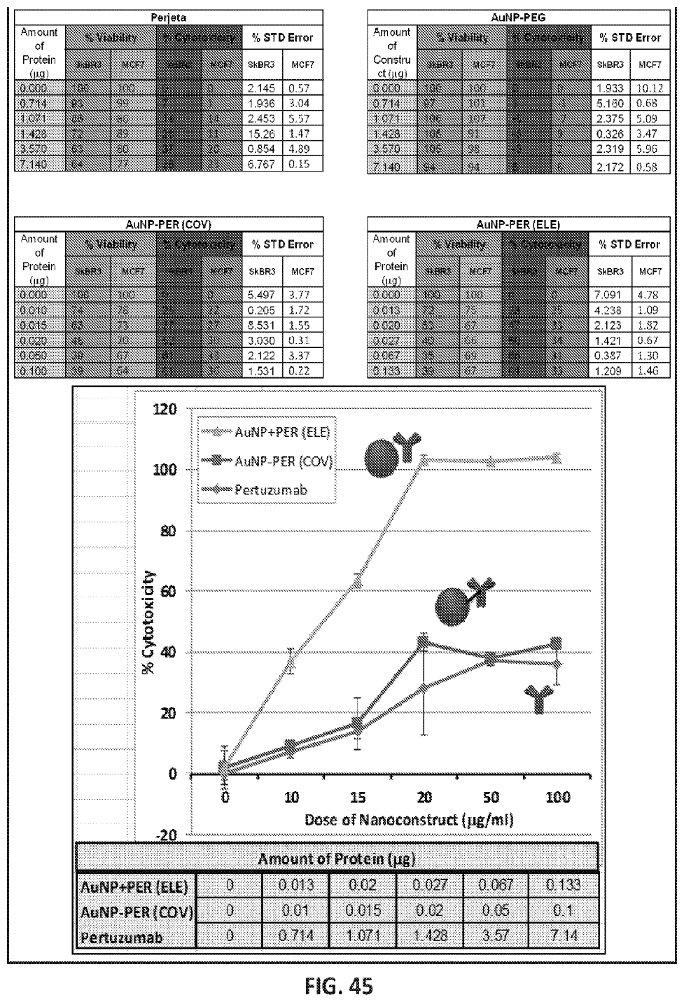

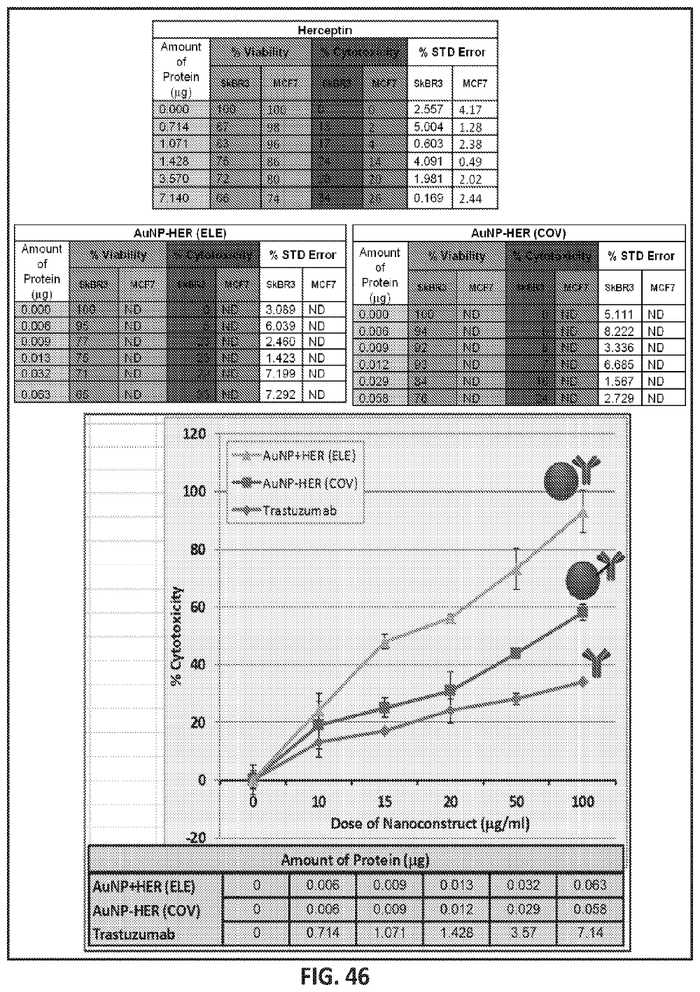

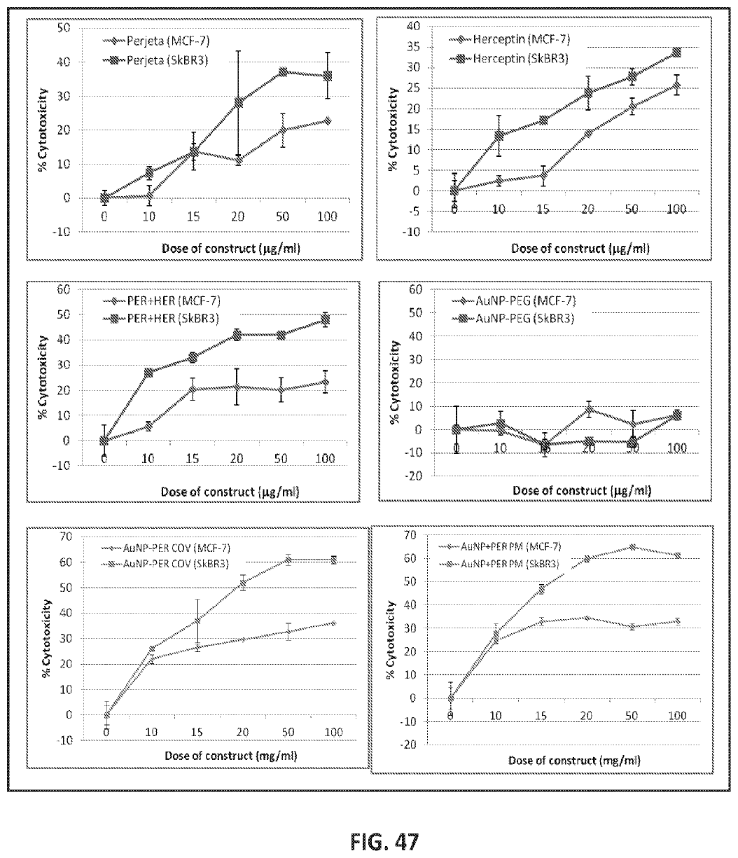

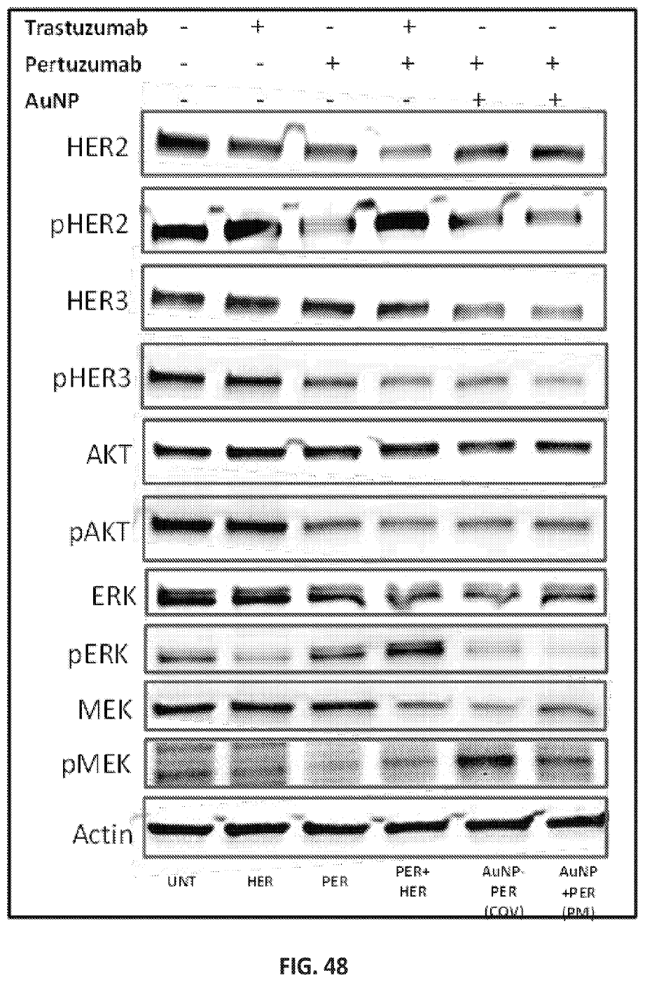

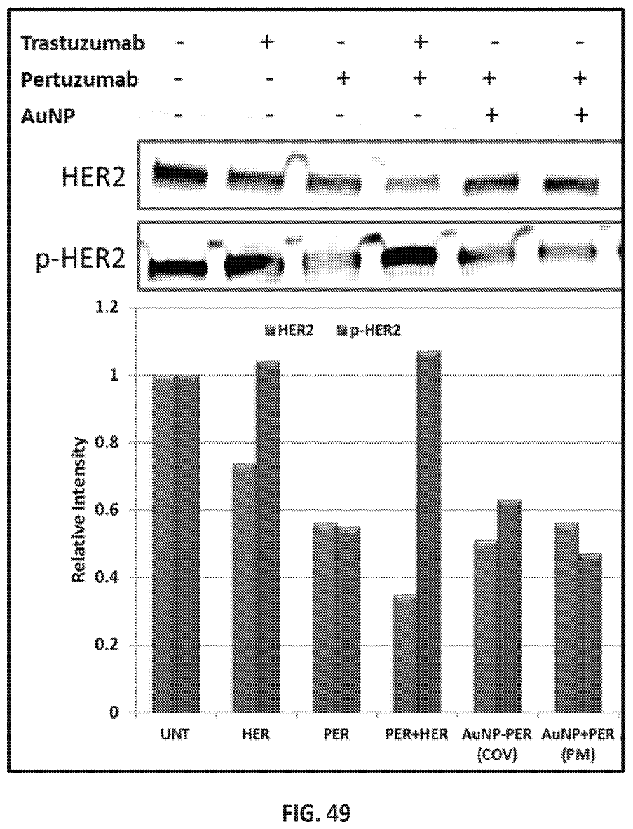

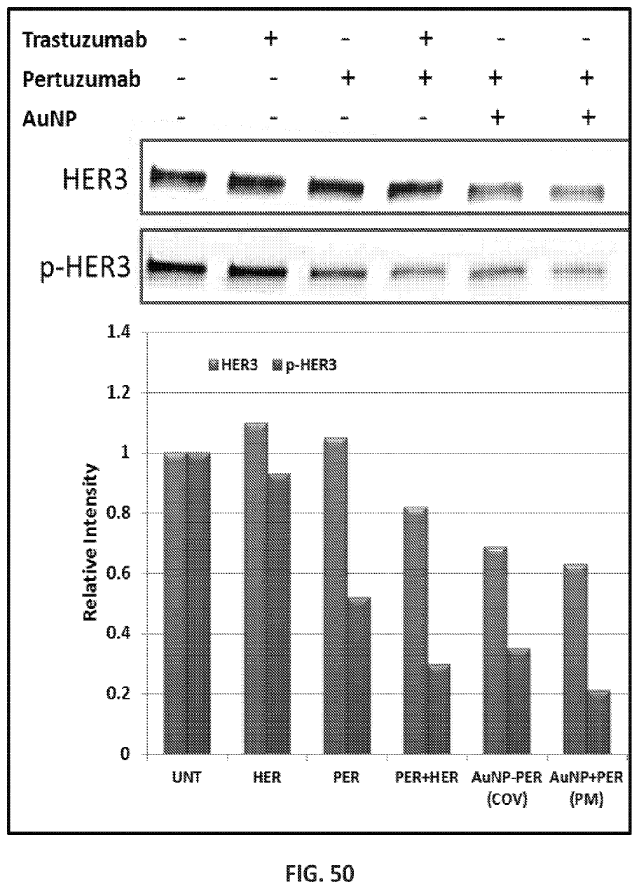

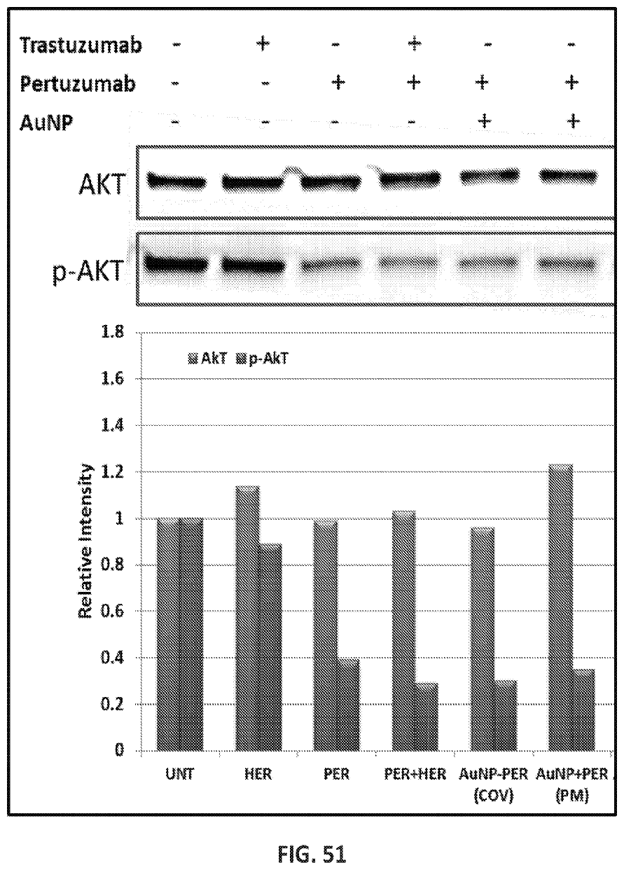

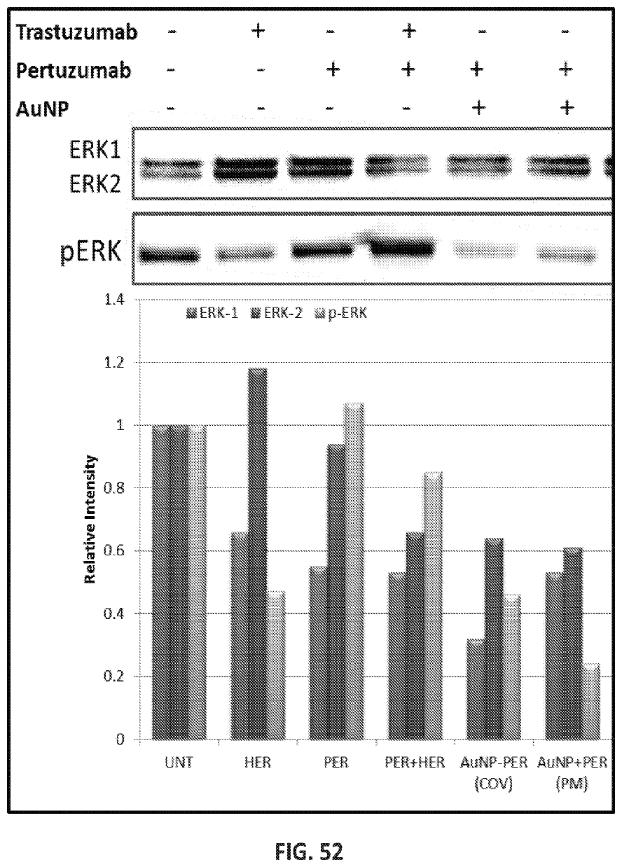

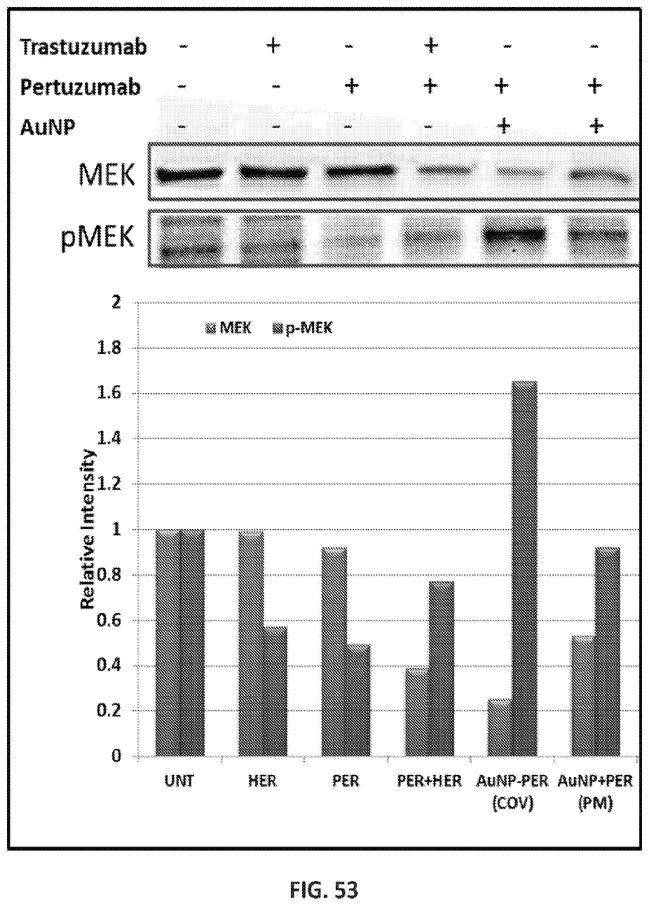

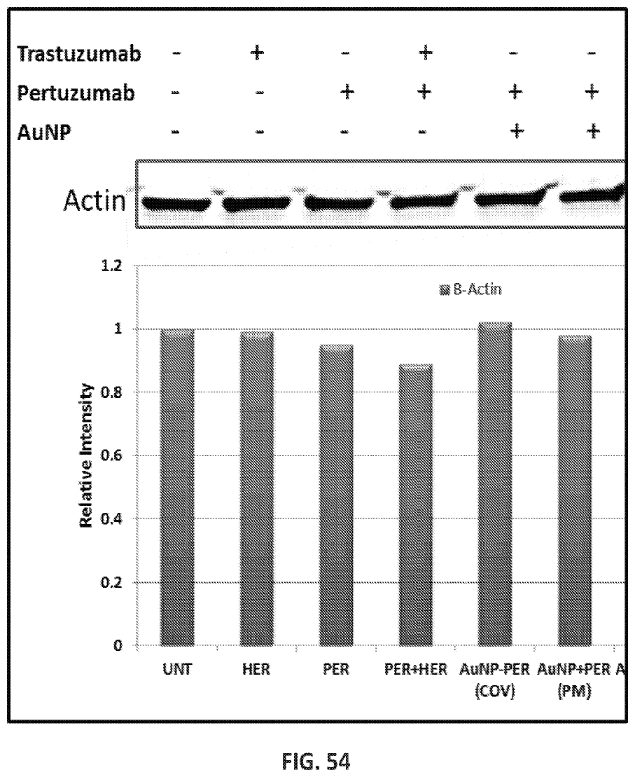

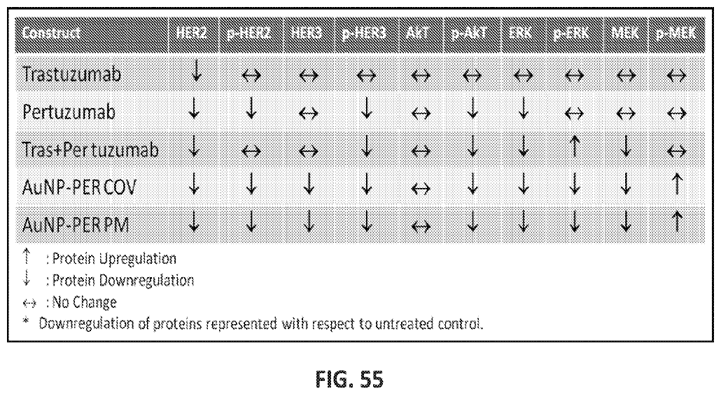

The linker weight was tested in experiments, and favorable weights provide favorable orientation. FIG. 42 represents when molecular weight PEG linker (mol. Wt. 2000) with end terminal carboxyl groups have favorable orientation to achieve maximum availability of antibody fragments Fa and Fb. FIG. 43 shows that when the molecular weight or chain length of linker was increased from 2000 to 3400, the linker would form a self-coil structure making the carboxyl groups unavailable for conjugation with a particular desired antibody. In FIG. 44, the ELISA binding study plots are given for antibody conjugated gold nanoparticles with 2000 and 3400 as the length of linker. Data points are given as mean absorbance. Antibody was diluted in a serial 10-fold dilution (stock of free antibody (Perjeta) used was 20 μg). FIGS. 45-55 provide physiological characterization examples that allow determination of functionality for predetermined selection of the same.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

What is claimed is:

1) A stable pharmaceutical formulation comprising a single antibody agent retaining its functionality in accordance with a predetermination of an a priori efficacy and electrostatically or covalently linked to a nanoparticle in a predetermined ratio in accordance with the a priori efficacy.

2) The formulation of claim 1, wherein the nanoparticle comprises a gold nanoparticle.

3) The formulation of claim 2, wherein said antibody agent is pertuzumab.

4) The formulation of claim 2, wherein said antibody agent is trastuzumab.

5) The formulation of claim 2, wherein said antibody agent is a pertuzumab antibody fragment.

6) The formulation of claim 2, wherein said antibody agent is a trastuzumab antibody fragment.

7) The formulation of claim 1, wherein said antibody agent is one of an antibody, an antibody fragment, affibody, a peptide, cyclic peptide, a toxin, a small molecule, a recombinant humanized monoclonal antibody, a rabbit antibody, a goat antibody, a mouse antibody, and an anti-hapten antibody.

8) The formulation of claim 1, wherein the diameter of said nanoparticle is from about 3 nm to about 80 nm.

9) The formulation of claim 1, wherein the diameter of said nanoparticle is from about 5 nm to about 15 nm.

10) The formulation of claim 1, wherein said linker linking said antibody and said nanoparticle comprises thiol or ethylene glycol.

11) The formulation of claim 11, wherein the linker is one of monoethylene glycol, diethylene glycol, and polyethylene glycol.

12) The formulation of claim 11, the linker is one of the chemical entities in the list comprising, but not limited to, thioctic acid, monothioctic acid, dithioctic acid, and trithioctic acid.

13) The formulation of claim 1, wherein the linker comprises a molecular weight of 3400 Da.

14) The formulation of claim 1, wherein the linker comprises a molecular weight of 2000 Da

15) A method of formation of nanoparticle conjugates for cancer diagnostics, imaging and therapy, wherein the method allows determination a priori the efficacy of the nanoparticle conjugate for treatment of disease, comprising synthesis of nanoparticles in solution, attachment of a linker to the nanoparticles, selective electrostatic or covalent coupling of a single antibody agent to the nanoparticles, and physicochemical characterization of the formed nanoconjugate.

16) The method of claim 15, wherein:
the synthesis comprises preparing citrate coated AuNPs in aqueous solution;
preparing AuNP-PEG-COOH in solution;
the attachment comprise attachment by activating carboxyl terminated AuNP-PEG-COOH using EDC/Sulfo-NHS and adding human antibody or fragment to a solution containing AuNP-PEG-COOH with an activation agent for covalent bonding or with no activation agent for electrostatic bonding.

17) The method of claim 16, wherein the activation agent comprises 1-Ethyl-3-[3-dimethylaminopropyl] carbodiimide hydrochloride/N-hydroxysulfosuccinimide ((EDC)/(Sulfo NHS)) in 4-morpholinoethanesulfonic acid (MES) buffer (pH=4.5)

18) The method of claim 17, wherein the human antibody or fragment is one of pertuzumab and trastuzumab.


SEQUENCE LISTING (6 sequences)

SEQ ID NO: 1 - protein, 214 aa, Artificial Sequence
  Trastuzumab, Anti-HER2 Light Chain 1
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 451 aa, Artificial Sequence
  Trastuzumab, Anti-HER2 Heavy Chain 1
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 214 aa, Artificial Sequence; identical to SEQ ID NO: 1
  Trastuzumab, Anti-HER2 Light Chain 2

SEQ ID NO: 4 - protein, 451 aa, Artificial Sequence; identical to SEQ ID NO: 2
  Trastuzumab, Anti-HER2 Heavy Chain 2

SEQ ID NO: 5 - protein, 214 aa, Artificial Sequence
  Amino acid sequence for Pertuzumab light chain
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 448 aa, Artificial Sequence
  Amino acid sequence for Pertuzumab heavy chain
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG